(19) United States
(12) Patent Application Publication
(10) Pub. No.: US 2025/0144382 A1
(43) Pub. Date: May 8, 2025

(54) EXPANDABLE APPOSITION ELEMENTS FOR SHUNTING CATHETERS

(71) Applicant: THERAHEART INC., Irvine, CA (US)

(72) Inventors: Rani Abdullah Mahmoudi, Costa Mesa, CA (US); James Wittel, Seattle, WA (US)

(21) Appl. No.: 18/933,837

(22) Filed: Oct. 31, 2024

Related U.S. Application Data
(60) Provisional application No. 63/596,126, filed on Nov. 3, 2023.

(51) Int. Cl. A61M 27/00; A61B 17/00; A61B 17/34; A61M 25/00

(52) U.S. Cl. CPC A61M 27/002; A61M 25/0074; A61B 2017/00247; A61B 2017/00336; A61B 17/3478; A61M 2025/0095; A61M 2210/125

(57) ABSTRACT

Some embodiments of the present disclosure are directed to systems, apparatus, and methods for creating a shunt in a patient. In some embodiments, a shunting catheter includes an apposition element for securing the catheter in the vasculature of the patient and a shunting element for creating the shunt in the patient.

### CROSS-REFERENCE TO RELATED APPLICATION

The present application claims the benefit of U.S. Provisional Application No. 63/596,126, entitled “EXPANDABLE APPOSITION ELEMENTS FOR SHUNTING CATHETERS,” filed on Nov. 3, 2023, which is incorporated by reference herein for all purposes in its entirety.

### TECHNICAL FIELD

Certain embodiments of the present disclosure relate to medical systems, apparatus, and methods for creating a shunt in a patient. More specifically, some embodiments of the present disclosure relate to medical systems, apparatus, and methods for creating a shunt on a cardiovascular system wall in a patient.

### BACKGROUND

Heart failure is a serious condition that occurs when a heart cannot pump enough blood and oxygen to support other organs in the body. Heart failure is classified according to left ventricular (LV) function as “heart failure with reduced ejection fraction (EF)” (HFrEF; EF<40%), “midrange EF” (HFmrEF; EF 40-49%), or “preserved EF” (HFpEF; EF≥50%). About half the patients with heart failure have HFpEF. HFpEF generally occurs when LV and left atrial filling pressures increase significantly during exercise, with an associated increase in pulmonary pressures leading to pulmonary congestion. Structural interventions to lower elevated either left or right atrial filling pressures are gaining attention.

Studies in heart failure show that lowering left atrial pressure may reduce cardiovascular events while improving functional capacity. The creation of an interatrial shunt has emerged as a therapy to decompress the left atrium in patients with acute and chronic left HF. As such, attention has turned toward the development of interatrial shunt devices (IASDs) as a means of reducing the detrimental increase in left-sided filling pressures with exercise in an effort to improve symptomatology. The IASDs may be used to treat various kinds of heart failure and/or other diseases that may result in too high of a pressure in the right atrium of a patient.

### SUMMARY

Many IASDs reside in the interatrial septum, with risk for right-to-left shunting and systemic embolization. Moreover, preservation of the interatrial septum is important with an increasing number of left-sided transseptal transcatheter interventions. Improved IASDs for safer and better procedures are needed.

In certain embodiments, a shunting catheter includes: a catheter shaft including a shaft lumen; a shunting element disposed in the shaft lumen at a first shunting element state and extended from the catheter shaft at a second shunting element state; and an apposition element disposed proximate to the shunting element, the apposition element disposed at the catheter shaft at a first apposition state and extending from the catheter shaft at a second apposition state.

In some embodiments, a method for creating a shunt includes: deploying a shunting catheter in a patient, the shunting catheter including: a catheter shaft including a shaft lumen; a shunting element disposed in the shaft lumen at a first shunting element state; and an apposition element disposed proximate to the shunting element, the apposition element disposed at the catheter shaft at a first apposition state; disposing the shunting catheter proximate to a target location of the patient; operating the apposition element to a second apposition state, wherein the apposition element extends from the catheter shaft at the second apposition state and stabilizes the shunting catheter in the patient proximate the target location; operating the shunting element to a second shunting element state, wherein the shunting element extends from the catheter shaft at the second shunting element state; puncturing, using the shunting element, an opening at the target location of the patient; and expanding, using the shunting element, the opening.

In some embodiments, a shunting catheter system includes: a shunting catheter, including: a catheter shaft including a shaft lumen; a shunting element disposed in the shaft lumen at a first shunting element state and extended from the catheter shaft at a second shunting element state, the shunting element including a puncture element; an apposition element disposed proximate to the shunting element, the apposition element disposed at the catheter shaft at a first apposition state and extending from the catheter shaft at a second apposition state; and an energy source connected to the shunting catheter; and a controller connected to the energy source and including a processor; the processor is configured to control the energy source to deliver ablation energy to a target location of a patient via the puncture element.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a diagram illustrating an exemplary clinical setting for treating a heart of the patient using a shunting catheter system, in accordance with embodiments of the present disclosure.

FIG. 2 is a schematic diagram illustrating an example of a shunting catheter to be deployed in a heart of a patient, in accordance with embodiments of the present disclosure.

FIG. 3 is a schematic diagram of a side view of an example of a shunting catheter, in accordance with embodiments of the present disclosure.

FIGS. 4A-4B are schematic diagrams of side views of an example of a shunting catheter, in accordance with embodiments of the present disclosure.

FIGS. 5A-5C are schematic diagrams of views of an example of a shunting element, in accordance with embodiments of the present disclosure.

FIGS. 6A-6B are schematic diagrams of side views of examples of puncture elements and inner members of ablation shafts, in accordance with embodiments of the present disclosure.

FIG. 7 is a schematic diagram of a perspective view of an example of an inner member of an ablation shaft, in accordance with embodiments of the present disclosure.

FIGS. 8A-8B are schematic diagrams of views of an example of an apposition element, in accordance with embodiments of the present disclosure.

FIGS. 9A-9B are schematic diagrams of views of another example of an apposition element, in accordance with embodiments of the present disclosure.

FIGS. 10A-10B are schematic diagrams of views of another example of an apposition element, in accordance with embodiments of the present disclosure.

FIG. 11 is a schematic diagram of a perspective view of another example of an apposition element, in accordance with embodiments of the present disclosure.

FIGS. 12A-12B are schematic diagrams of side views of an example of a shunting catheter, in accordance with embodiments of the present disclosure.

FIGS. 13A-13C are schematic diagrams of perspective views of an example of a shunting catheter, in accordance with embodiments of the present disclosure.

FIGS. 13D-13F are schematic diagrams of cross-sectional views of a apposition element and a catheter shaft of the shunting catheter along lines 13D-13D, 13E-13E, and 13F-13F, respectively, of FIG. 13A, in accordance with embodiments of the present disclosure.

FIG. 14A is a schematic diagram of a perspective view of an example of a shunting catheter, in accordance with an embodiment of the present disclosure.

FIGS. 14B-14E are schematic diagrams of cross-sectional views of a apposition element and a catheter shaft of the shunting catheter along lines 14B-14B, 14C-14C, 14D-14D, and 14E-14E respectively, of FIG. 14A, in accordance with embodiments of the present disclosure.

FIGS. 15A-15B are schematic diagrams of side views of an example of a shunting catheter, in accordance with certain embodiments of the present disclosure.

FIGS. 16A-16B are schematic diagrams of side views of another example of a shunting catheter, in accordance with certain embodiments of the present disclosure.

FIG. 17 is a flow diagram illustrating an example method of creating a shunt in a patient, in accordance with embodiments of the present disclosure.

FIG. 18 is a flow diagram illustrating another example method of creating a shunt in a patient, in accordance with embodiments of the present disclosure.

While the present disclosure is amenable to various modifications and alternative forms, specific embodiments have been shown by way of example in the drawings and are described in detail below. The present disclosure, however, is not to limit the present disclosure to the particular embodiments described. On the contrary, the present disclosure is intended to cover all modifications, equivalents, and alternatives falling within the scope of the present disclosure as defined by the appended claims.

### DETAILED DESCRIPTION

The following detailed description is exemplary in nature and is not intended to limit the scope, applicability, or configuration of the present disclosure in any way. Rather, the following description provides some practical illustrations for implementing exemplary embodiments of the present disclosure. Examples of constructions, materials, and/or dimensions are provided for selected elements. Those skilled in the art will recognize that many of the noted examples have a variety of suitable alternatives.

Unless otherwise indicated, all numbers expressing feature sizes, amounts, and physical properties used in the specification and claims are to be understood as being modified in all instances by the term “about.” Accordingly, unless indicated to the contrary, the numerical parameters set forth in the foregoing specification and attached claims are approximations that can vary depending upon the desired properties sought to be obtained by those skilled in the art utilizing the teachings disclosed herein. The use of numerical ranges by endpoints includes all numbers within that range (for example, 1 to 5 includes 1, 1.5, 2, 2.75, 3, 3.80, 4, and 5) and any number within that range.

Although illustrative methods may be represented by one or more drawings (for example, flow diagrams, communication flows, etc.), the drawings should not be interpreted as implying any requirement of, or particular order among or between, various steps disclosed herein. However, some embodiments may require certain steps and/or certain orders between certain steps, as may be explicitly described herein and/or as may be understood from the nature of the steps themselves (for example, the performance of some steps may depend on the outcome of a previous step). Additionally, a “set,” “subset,” or “group” of items (for example, inputs, algorithms, data values, etc.) may include one or more items and, similarly, a subset or subgroup of items may include one or more items. A “plurality” means more than one.

As used herein, the term “based on” is not meant to be restrictive, but rather indicates that a determination, identification, prediction, calculation, and/or the like, is performed by using, at least, the term following “based on” as an input. For example, predicting an outcome based on a particular piece of information may additionally, or alternatively, base the same determination on another piece of information. In some embodiments, the term “receive” or “receiving” means obtaining from a data repository (for example, database), from another system or service, from another software, or from another software component in a same software. In certain embodiments, the term “access” or “accessing” means retrieving data or information, and/or generating data or information.

There are various approaches for creating an interatrial shunt, which is a connection or gateway between the left and right atria of a patient's heart for blood to flow through. In some embodiments, examples of interatrial shunt devices (IASDs) include implants or shunting catheters. For example, devices reside in the interatrial septum, with risk for right-to-left shunting and systemic embolization. In some examples, preservation of the interatrial septum is important with an increasing number of left-sided transseptal transcatheter interventions. Improved IASDs for safer and better procedures are needed. At least some embodiments of the present disclosure are directed to a shunting catheter for deployment through a patient's coronary sinus (CS) for creating a shunt between the CS and the patient's left atrium (LA).

A patient's CS ostium may have a diameter of from about 10 mm to about 20 mm. As the CS is a relatively small vessel, at least some embodiments of the present disclosure include features of a shunting catheter that helps protect a patient's vessels during deployment and/or elements for stabilizing the catheter during the procedure. In embodiments, the shunting catheter includes a catheter shaft, a shunting element, and an apposition element. The apposition element stabilizes the shunting catheter in the patient during the procedure. In some embodiments, the catheter shaft is made of flexible materials that bends according to the anatomy of the CS to conform to the shape of the patient's CS. In some embodiments, the catheter shaft includes a stabilizing element such as distal tip that has a curve (for example, a pre-existing curve) conforming to the shape of a patient's CS to help stabilize the catheter and minimize potential damage to the vessel wall of a patient's CS.

In certain embodiments, the shunting element is disposed in a shaft lumen of the catheter shaft at a first shunting element state and is extended from the catheter shaft at a second shunting element state. In some embodiments, a shunt is formed by creating an opening between the patient's CS and LA. In some embodiments, a shunt is formed by creating an opening between the patient's RA and LA. In certain embodiments, the shunting catheter is inserted through the patient's superior vena cava (SVC) via a transjugular approach. In certain embodiments, the shunting catheter is inserted through the patient's inferior vena cava (IVC) via a transfemoral approach.

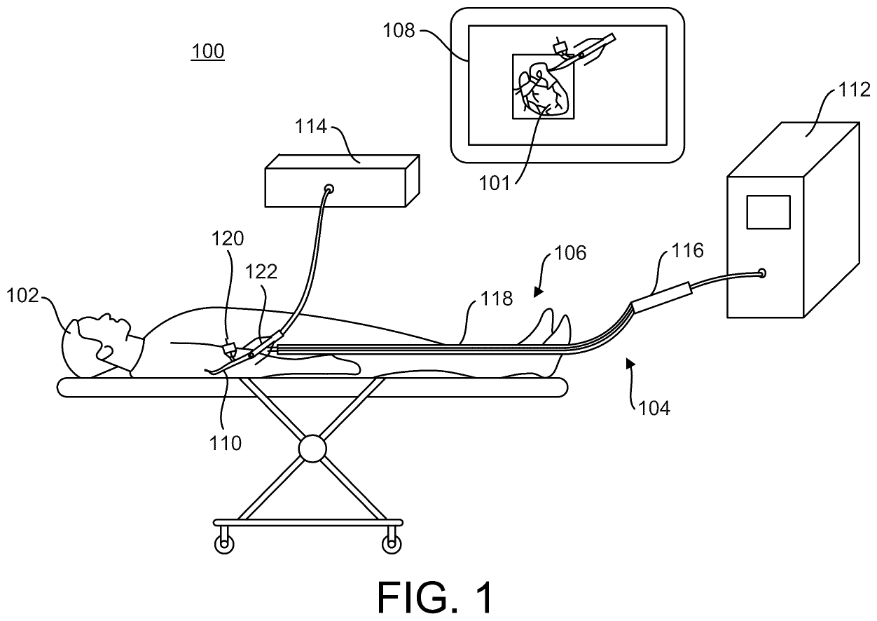

FIG. 1 is a diagram illustrating an exemplary clinical setting 100 for treating a heart 101 of a patient 102 using a shunting catheter system 104, in accordance with embodiments of the present disclosure. In certain embodiments, the shunting catheter system 104 includes a shunting device 106. As will be appreciated by the skilled artisan, the clinical setting 100 may have other components and arrangements of components that are not shown in FIG. 1. In some embodiments, the shunting catheter system 104 includes or is coupled to an imaging system (for example, an X-ray system), which may include one or more visualization elements and a display 108. In some embodiments, one or more visualization elements may be disposed on the shunting device 106. In certain embodiments, the imaging system can help guide a physician's operation of the shunting device 106 during a procedure.

According to certain embodiments, the shunting device 106 includes a shunting catheter 110, a controller 112, and an energy source 114 (for example, a generator). In some embodiments, the controller 112 is configured to control functional aspects of the shunting device 106. In some embodiments, the controller 112 is configured to control the energy source 114 to deliver energy to the shunting catheter 110. In certain embodiments, the controller 112 may be connected to the one or more visualization elements to facilitate positioning of the shunting catheter 110 in a patient's heart during procedure. In some embodiments, the energy source 114 is connected to the controller 112. In some embodiments, the energy source 114 may be integrated with the controller 112.

According to some embodiments, the shunting device 106 includes a handle 116, a catheter shaft 118, and a shunting element 120. In certain embodiments, the handle 116 is configured to be operated by a user to position the shunting element 120 at a target shunting location. In certain embodiments, the shunting element 120 includes a puncture element (for example, a puncture needle) configured to puncture through a vessel wall. In certain embodiments, the shunting element 120 is connected to the energy source 114 to provide shunting. For example, the shunting element 120 receives energy from the energy source 114 to deliver energy (for example, ablation energy, such as radiofrequency (RF) energy, phased RF energy, cryogenic energy, thermal energy, pulse energy, laser energy, microwave energy, ultrasound energy, and/or the like) to the target location (for example, a target tissue) at a cardiovascular system (for example, a circulatory system) wall. In certain embodiments, the energy source 114 provides energy in a first form (for example, electrical energy) to the shunting element 120, and the shunting element 120 delivers the ablation energy to the target location in a second form (for example, radiofrequency (RF) energy, phased RF energy, cryogenic energy, thermal energy, pulse energy, laser energy, microwave energy, ultrasound energy, and/or the like).

According to certain embodiments, during deployment, the shunting device 106 including a portion of the catheter shaft 118 enters through a patient's CS ostium. The shunting device 106 may then be oriented through one or more mechanisms in the patient's CS, as will be discussed in more detail below. In some embodiments, in order to conform to the shape of the patient's CS, the catheter shaft 118 is made of flexible materials and/or has a structure that may bend according to the anatomy of the CS. In certain embodiments, during deployment, the puncture element creates an opening at a target tissue (for example, a vessel wall), and then the shunting element 120 enlarges the opening at the target tissue.

In certain embodiments, the controller 112 controls the delivery of ablation energy (for example, radiofrequency (RF) energy, phased RF energy, cryogenic energy, thermal energy, pulse energy, laser energy, microwave energy, ultrasound energy, and/or the like) via the shunting element 120 after and/or when the opening is generated by the puncture element and/or the shunting element 120.

In certain embodiments, the shunting element 120 includes a cage having a plurality of movable struts. In certain embodiments, the struts are configured to receive energy from the energy source and deliver ablation energy to a target location of a patient. In certain embodiments, one or more of the struts carry an electrode, and the electrode is configured to receive energy from the energy source and deliver ablation energy to a target location of a patient. In certain embodiments, the struts comprise braided wires. In certain embodiments, the shunting element comprises a laser-cut tube, and the struts are disposed at an end of the laser-cut tube. In some embodiments, the struts are self-expandable. In certain embodiments, the struts are expandable via an actuator (for example, an inflatable balloon) carried within the cage. In certain embodiments, the struts are constructed of at least one material selected from a group consisting of nitinol, stainless steel, titanium, platinum-iridium, and cobalt-chromium.

In certain embodiments, the shunting catheter 110 includes an apposition element 122 disposed proximate to the shunting element 120. In some embodiments, the apposition element 122 is disposed at the catheter shaft 118 (or pressed against the catheter shaft 118; more specifically, for example, within an outer shaft) at a first apposition state. In some embodiments, the apposition element 122 extends from the catheter shaft 118 (more specifically, for example, outside of the outer shaft) at a second apposition state. In certain embodiments, the apposition element 122 can appose to a cardiovascular system wall (for example, the front wall or back wall of the CS, a left atrium wall, a right atrium wall, etc.) at the second apposition state, for example, to help position and/or stabilize the shunting element 120. In certain embodiments, the apposition element 122 includes a plurality of movable struts that together define an expandable cage. In certain embodiments, the apposition element 122 includes a braid structure. In some embodiments, the apposition element 122 may include a nitinol braid that can be held within the catheter shaft 118 or the outer shaft. In certain embodiments, after deployment and stabilization of the catheter shaft 118, the shunting element 120 and the puncture element may then be deployed. In some embodiments, the shunting element 120 is configured to deliver ablation energy to target tissues for creating a shunt in the patient's CS.

According to some embodiments, various components (for example, the controller 112) of the shunting catheter system 104 may be implemented on one or more computing devices. In certain embodiments, a computing device may include any type of computing device suitable for implementing embodiments of the disclosure. Examples of computing devices include specialized computing devices or general-purpose computing devices such as workstations, servers, laptops, portable devices, desktop, tablet computers, hand-held devices, general-purpose graphics processing units (GPGPUs), and the like, all of which are contemplated within the scope of FIG. 1 with reference to various components of the shunting catheter system 104.

In some embodiments, a computing device (for example, the controller 112) includes a bus that, directly and/or indirectly, couples the following devices: a processor, a memory, an input/output (I/O) port, an I/O component, and a power supply. Any number of additional components, different components, and/or combinations of components may also be included in the computing device. The bus represents what may be one or more busses (such as, for example, an address bus, data bus, or combination thereof). Similarly, in some embodiments, the computing device may include a number of processors, a number of memory components, a number of I/O ports, a number of I/O components, and/or a number of power supplies. Additionally, any number of these components, or combinations thereof, may be distributed and/or duplicated across a number of computing devices. In some embodiments, various components or parts of components (for example, controller 112, shunting catheter 110, etc.) can be integrated into a physical device.

In some embodiments, the shunting catheter system 104 includes one or more memories (not illustrated). The one or more memories includes computer-readable media in the form of volatile and/or nonvolatile memory, transitory and/or non-transitory storage media and may be removable, nonremovable, or a combination thereof. Media examples include Random Access Memory (RAM); Read Only Memory (ROM); Electronically Erasable Programmable Read Only Memory (EEPROM); flash memory; optical or holographic media; magnetic cassettes, magnetic tape, magnetic disk storage or other magnetic storage devices; data transmissions; and/or any other medium that can be used to store information and can be accessed by a computing device such as, for example, quantum state memory, and/or the like. In some embodiments, the one or more memories store computer-executable instructions for causing a processor (for example, the controller 112) to implement aspects of embodiments of system components discussed herein and/or to perform aspects of embodiments of methods and procedures discussed herein.

Computer-executable instructions may include, for example, computer code, machine-useable instructions, and the like such as, for example, program components capable of being executed by one or more processors associated with a computing device. Program components may be programmed using any number of different programming environments, including various languages, development kits, frameworks, and/or the like. Some or all of the functionality contemplated herein may also, or alternatively, be implemented in hardware and/or firmware.

In some embodiments, the memory may include a data repository that may be implemented using any one of the configurations described below. A data repository may include random access memories, flat files, XML files, and/or one or more database management systems (DBMS) executing on one or more database servers or a data center. A database management system may be a relational DBMS (RDBMS), hierarchical DBMS (HDBMS), multidimensional DBMS (MDBMS), object oriented DBMS (ODBMS or OODBMS) or object relational DBMS (ORDBMS), and/or the like. The data repository may be, for example, a single relational database. In some cases, the data repository may include a plurality of databases that can exchange and aggregate data by a data integration process or software application. In an exemplary embodiment, at least part of the data repository may be hosted in a cloud data center. In some cases, a data repository may be hosted on a single computer, a server, a storage device, a cloud server, or the like. In some other cases, a data repository may be hosted on a series of networked computers, servers, or devices. In some cases, a data repository may be hosted on tiers of data storage devices including local, regional, and central.

Various components of the shunting catheter system 104 can communicate via or be coupled to via a communication interface, for example, a wired or wireless interface. The communication interface includes, but is not limited to, any wired or wireless short-range and long-range communication interfaces. The wired interface can use cables, umbilicals, and the like. The short-range communication interfaces may be, for example, local area network (LAN), interfaces conforming to known communications standards, such as Bluetooth™ standard, IEEE 802 standards (for example, IEEE 802.11), a ZigBee™ or similar specification, such as those based on the IEEE 802.15.4 standard, or other public or proprietary wireless protocol. The long-range communication interfaces may be, for example, wide area network (WAN), cellular network interfaces, satellite communication interfaces, etc. The communication interface may be either within a private computer network, such as intranet, or on a public computer network, such as the internet. Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

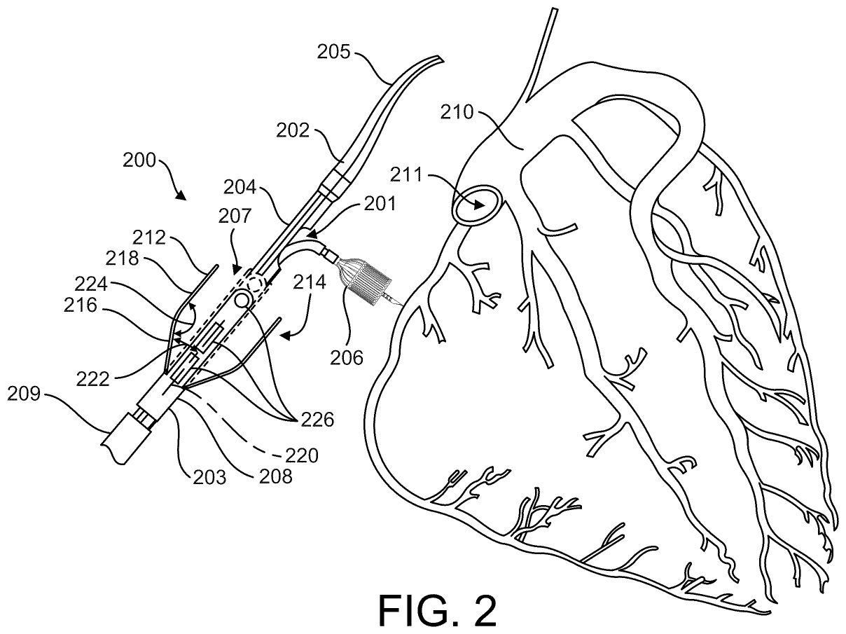

FIG. 2 is a schematic diagram illustrating an example of a shunting device 200 to be deployed in a heart of a patient, in accordance with embodiments of the present disclosure. FIG. 2 is merely an example. One of the ordinary skilled in the art would recognize many variations, alternatives, and modifications. As shown, the shunting device 200 includes a shunting catheter 202 to be deployed to a patient's coronary sinus (CS) 210 via the CS ostium 211. In certain embodiments, the shunting catheter 202 is deployed to a patient's right atrium (RA) via the inferior vena cava (IVC). In some embodiments, the shunting catheter 202 includes a catheter shaft 204, a shunting element 206, and an apposition element 208. In certain embodiments, the catheter shaft 204 has a curve at its distal end 205. In some embodiments, as illustrated, the shunting element 206 is extended from the catheter shaft 204 at a state to provide shunting (for example, a second shunting element state different from a first shunting element state). In certain examples, the shunting element 206 forms an angle greater than 10 degrees from the distal end 205 of the catheter shaft 204. In some examples, the shunting element 206 forms an angle greater than 30 degrees from the distal end 205 of the catheter shaft 204. In some embodiments, the shunting element 206 forms an angle proximate to 90 degrees from the catheter shaft 204. In some embodiments, the shunting element 206 forms an angle in the range of 10 degrees to 120 degrees from the catheter shaft 204.

In some embodiments, the shunting element 206 includes a cage having a plurality of movable struts. In certain embodiments, the struts are configured to receive energy from the energy source and deliver ablation energy to a target location of a patient. In certain embodiments, one or more of the struts carry an electrode, and the electrode is configured to receive energy from the energy source and deliver ablation energy to a target location of a patient. In certain embodiments, the struts comprise braided wires. In certain embodiments, the shunting element 206 comprises a laser-cut tube, and the struts are disposed at an end of the laser-cut tube. In some embodiments, the struts are self-movable (for example, by being made of a shape memory material and set in the expanded state) and the shunting element 206 is thereby self-expandable. In certain embodiments, the struts are expandable via an actuator (for example, an inflatable balloon) carried within the cage. In certain embodiments, the struts are constructed of at least one material selected from a group consisting of nitinol, stainless steel, titanium, platinum-iridium, and cobalt-chromium.

In some embodiments, the apposition element 208 is disposed at the catheter shaft 204 (or pressed against the catheter shaft 204; more specifically, for example, within an outer shaft 209 movable outside of the catheter shaft 204 and the apposition element 208) at a first apposition state (shown in phantom lines). In some embodiments, the apposition element 208 extends from the catheter shaft 204 (more specifically, for example, outside of the outer shaft) at a second apposition state (shown in solid lines). In certain embodiments, the apposition element 208 can appose to a cardiovascular system wall (for example, the front wall or back wall of the CS, a left atrium wall, a right atrium wall, etc.) at the second apposition state, for example, to help position and/or stabilize the shunting element 206.

In some embodiments and as illustrated, the apposition element 208 includes a first end 203 and a second end 207, and the first end 203 is secured to the catheter shaft 204 and the second end 207 is disposed apart from the catheter shaft 204. In other embodiments, the first end 203 is movable relative to the catheter shaft 204 and the second end 207 is secured to the catheter shaft 204.

In certain embodiments, the apposition element 208 includes a plurality of movable struts 212 that together define an expandable cage 214. In some embodiments, one or more of the movable struts 212 includes a first strut portion 216 and a second strut portion 218, the first strut portion 216 and an axis 220 defined by the catheter shaft 204 form an expansion angle 222 in the second apposition state, and the expansion angle 222 is greater than zero degrees. In certain embodiments, the expansion angle 222 is greater than 15 degrees. In some embodiments, the first strut portion 216 and the second strut portion 218 form a strut angle 224 greater than zero degrees in the second apposition state. In certain embodiments, one or more of the movable struts 212 includes one or more holes 226. In some embodiments, the holes 226 of the struts 212 and/or the spaces between adjacent struts 212 allow blood flow through the apposition element 208, thus reducing the risk of thrombus formation caused by any occlusion in the vessel.

In some embodiments, the apposition element 208 has a height from the catheter shaft 204 that is the maximum distance of the apposition element 208 from the outer surface of the catheter shaft 204. In certain embodiments, the apposition element 208 has a first height from the catheter shaft 204 at the first apposition state and a second height from the catheter shaft 204 at the second apposition state, and the second height is greater than the first height. In some embodiments, the second height is at least two times the first height. In certain embodiments, the first height is in a range of 0 mm to 1 mm. In certain embodiments, the second height is in a range of 4 mm to 10 mm.

In some embodiments and as illustrated, the apposition element 208 is disposed substantially symmetrically around the catheter shaft 204, more specifically the struts 212 are disposed at equal angles around the catheter shaft 204. In other embodiments, the apposition element 208 is disposed substantially non-symmetrically around the catheter shaft 204, more specifically the struts 212 are disposed at unequal angles around the catheter shaft 204.

In certain embodiments, the movable struts 212 comprise braided wires. In certain embodiments, the apposition element 208 comprises a laser-cut component. In some embodiments, the struts are self-movable (for example, by being made of a shape memory material and set in the expanded state) and the apposition element 208 is thereby self-expandable. In certain embodiments, the struts are movable via an actuator (for example, an inflatable balloon) carried within the cage. In certain embodiments, the struts are constructed of at least one material selected from a group consisting of nitinol, stainless steel, titanium, platinum-iridium, and cobalt-chromium.

In certain embodiments, the apposition element 208 is longitudinally offset from a shaft opening 201 in the catheter shaft 204 through which the shunting element 206 extends. In some embodiments, the apposition element 208 is longitudinally offset from the opening 201 in the catheter shaft 204 by up to 20 mm. In other embodiments, the apposition element 208 is longitudinally aligned with the opening 201 in the catheter shaft 204.

In some embodiments, the catheter shaft 204 is made of flexible material that may curve with the anatomy of the patient's CS 210. In certain embodiments, for example, the catheter shaft 204 may include polyether block amide, nylon, silicone, or a combination thereof. In some embodiments, the catheter shaft 204 may be a multi-layered and multi-material component. In some examples, the catheter shaft 204 is reinforced with a braid and/or can have an etched or casted liner. In certain embodiments, the braid for reinforcing the catheter shaft 204 may be made of nitinol. In some embodiments, the liner may be made from polyetheretherketone (PEEK), polytetrafluoroethylene (PTFE), copolymers of polyamide and polyether, or a combination thereof. In some embodiments, the catheter shaft 204 is coated for lubricity with a hydrophilic coating, or other types of coating suitable for coating a catheter shaft as known by a skilled person in the art.

In some embodiments, the shunting catheter 202 has a diameter of from about 2 mm to about 6 mm. In certain embodiments, the shunting catheter 202 has a diameter from about 2.5 mm to about 5 mm. In some embodiments, the shunting catheter 202 has a diameter from about 3 mm to about 4 mm. In certain embodiments, the shunting catheter 202 may have a diameter allowing it to pass through vessels and parts of the cardiovascular system to reach a target location.

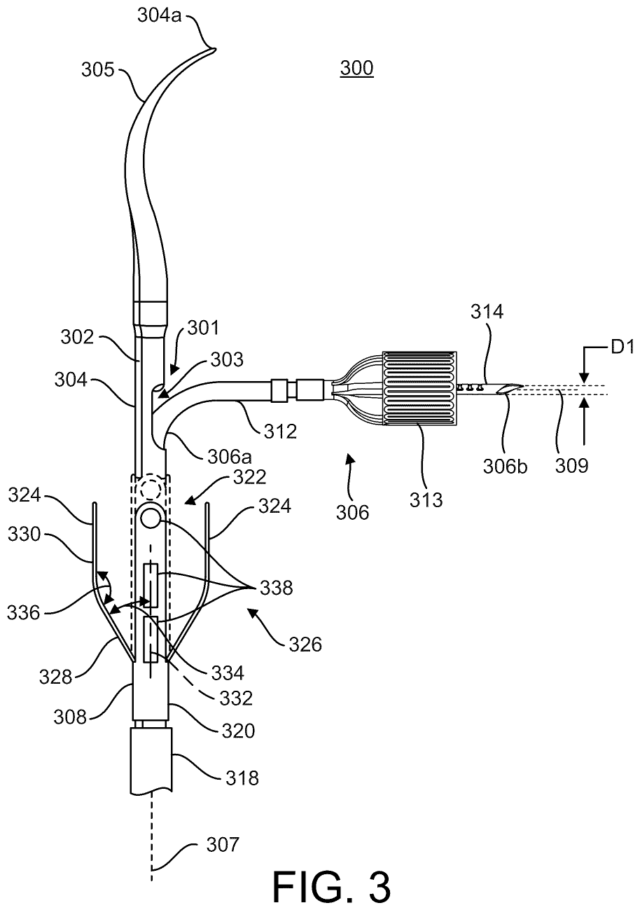

FIG. 3 is a schematic diagram of a side view of an example of a shunting device 300, in accordance with embodiments of the present disclosure. FIG. 3 is merely an example. One of the ordinary skilled in the art would recognize many variations, alternatives, and modifications. As shown, the shunting device 300 includes a shunting catheter 302. In some embodiments, the shunting catheter is configured to be delivered through a patient's coronary sinus (CS). In some embodiments, the shunting catheter 302 includes a catheter shaft 304, a shunting element 306, and an apposition element 308.

According to some embodiments, the shunting catheter 302 may be inserted through a small vein in the patient's body, and then tracked to the patient's right atrium (RA). In certain embodiments, once the shunting catheter 302 is in the patient's RA, the shunting catheter 302 may be maneuvered into the CS ostium to gain alignment in the CS at a target location of on a wall between the patient's CS and LA.

According to certain embodiments, the catheter shaft 304 is made of flexible material that may curve with the anatomy of the patient's CS. In certain embodiments, the catheter shaft 304 may include polyether block amide, nylon, silicone, and/or a combination thereof. In some instances, the catheter shaft 304 may be a multi-layered and multi-material component. In some instances, the shunting catheter 302 may be made from multiple materials that are reflow soldered together. In certain instances, the shunting catheter 302 may be made from multiple materials that are bonded together with an over mold. In certain embodiments, there may be a portion of the shunting catheter 302 that houses other components of the shunting device 300 that are configured to interact with the patient's anatomy.

In some embodiments, the catheter shaft 304 is reinforced with a braid, metal coil, or laser cut hypotube, and can have an etched or casted liner. In certain embodiments, the braid for reinforcing the catheter shaft 304 may be made of materials such as nitinol, stainless steel, liquid crystal polymer (LCP), Kevlar, PEEK, PET, carbon fiber, fiberglass, tungsten, platinum, iridium, ultra-high molecular weight polyethylene (UHMWPE), and others. In some embodiments, the liner may be made from polyetheretherketone (PEEK), polytetrafluoroethylene (PTFE), copolymers of polyamide and polyether, or a combination thereof. In certain embodiments, the catheter shaft 304 may be injection molded or extruded. In some embodiments, the catheter shaft 304 is coated for lubricity with a hydrophilic coating, or other types of coating suitable for coating a catheter shaft as known by a skilled person in the art.

In certain embodiments, the catheter shaft 304 may have multiple lumens. In embodiments, the multiple lumens may allow for the exchange and movement of various parts (for example, the shunting element 306, the apposition element 308) during deployment and/or shunting. In certain embodiments, the shunting catheter 302 is used to gain access into a patient's CS, the shunting catheter 302 including multiple lumens to gain access into the patient's LA.

According to some embodiments, the catheter shaft 304 has a distal end 304a and a proximal end (not shown). In some embodiments, the catheter shaft 304 may include a stabilizing element such as distal tip 305 at the distal end 304a that has a curve (for example, a pre-existing curve), for example, a curve conforming to the anatomy of a patient's CS. In embodiments, the distal tip 305 may help with navigation when inserting the shunting catheter 302 into the patient's CS. In certain embodiments, the distal tip 305 may allow for proper positioning of the shunting catheter 302 during shunting. In some instances, the distal tip 305 may be made of a different material than other parts of the catheter shaft 304. In some instances, for example, the distal tip 305 may be made of a material more flexible than the material of other parts of the catheter shaft 304. In some embodiments, the distal tip 305 may be injection molded or machined to have a unique geometry (for example, a curve) for better stabilizing the catheter shaft 304 during deployment.

According to some embodiments, the distal tip 305 may have a length of from about 5 mm to about 85 mm. In certain embodiments, the catheter shaft 304 includes a shaft opening 303. In some embodiments, a portion of the catheter shaft 304 between the shaft opening 303 and the distal end 304a has a curve. In some embodiments, the catheter shaft 304 defines a first axis 307, and the shunting element 306 defines a second axis 309 at the second shunting element state. In certain embodiments, the second axis 309 and the first axis 307 form an angle greater than zero degree. In certain embodiments, the second axis 309 and the first axis 307 form an angle greater than 10 degrees.

According to certain embodiments, the catheter shaft 304 includes a shaft lumen 301, and the shunting element 306 is disposed in the shaft lumen 301 at a first shunting element state (for example, during deployment, during deployment to position of the shunting element 306). In certain embodiments, the shunting element 306 includes a distal end 306a and a proximal end 306b. In some embodiments, the shunting element 306 includes a shunting shaft 312, a shunting mechanism 313, and a puncture element 314. In certain embodiments, the shunting shaft 312 has a pre-determined curve. In certain embodiments, the shunting mechanism 313 is extended from the catheter shaft 304 at the proximal end 306b of the shunting element 306 at a second state (for example, a shunting state). In some instances, the shunting element 306 extends from the catheter shaft 304 through the shaft opening 303. In certain instances, the puncture element 314 has a diameter (d1) in the range of about 2 millimeters to about 5 millimeters. In some embodiments, once the shunting catheter 302 is in position after deployment, the puncture element 314 may be used to puncture through the wall between a patient's CS and LA.

According to certain embodiments, an energy source coupled to the shunting catheter 302 may provide energy (for example, electrical energy) to the shunting catheter 302, and the shunting catheter 302 may generate and deliver ablation energy (for example, radiofrequency (RF) energy, phased RF energy, cryogenic energy, thermal energy, pulse energy, laser energy, microwave energy, ultrasound energy, and/or the like) to a target location of the patient.

According to some embodiments, the puncture element 314 is disposed at the distal end 306b of the shunting element 306. In certain embodiments, the shaft opening 303 is not at the distal end 304a of the catheter shaft 304. In certain embodiments, the puncture element 314 has a configuration of regular trocar point, regular taper point, regular taper cutting, regular reverse cutting edge, regular diamond point, regular conventual cutting edge, regular blunt taper point, premium lancet point, premium diamond point, or premium cutting edge. In certain embodiments, the puncture element 314 is made of materials including nitinol, stainless steel, cobalt chromium, aluminum, and/or a combination thereof.

In some embodiments, the shunting element 306 is configured to deliver ablation energy to a target tissue during shunting. In certain embodiments, the ablation energy delivered by the shunting element 306 may include radiofrequency (RF) energy, phased RF energy, cryogenic energy, thermal energy, pulse energy, laser energy, microwave energy, ultrasound energy, and/or the like. In certain embodiments, the energy delivered by the shunting element 306 punctures through tissue surrounding the target location to create an opening at the target location. In some embodiments, the energy delivered by the shunting element 306 ablates tissue surrounding the target location to solidify an opening at the target location. In certain embodiments, delivering energy via the shunting element 306 helps prevent tissue regrowth around the created shunt after the procedure. In other embodiments, the shunting element 306 does not deliver ablation energy to the target tissue during shunting.

According to some embodiments, the shunting catheter 302 further includes an outer shaft 318 disposed outside of at least a part of the catheter shaft 304 during deployment. In some embodiments, the outer shaft 318 is made of flexible material that may curve with the anatomy of the patient's CS. In certain embodiments, for example, the outer shaft 318 may include polyether block amide, nylon, silicone, or a combination thereof. In some instances, the outer shaft 318 may be a multi-layered and multi-material component.

In some examples, the outer shaft 318 is reinforced with a braid and can have an etched or casted liner. In some embodiments, the braid for reinforcing the catheter shaft 304 may be made of nitinol. In certain embodiments, the liner may be made from polyetheretherketone (PEEK), polytetrafluoroethylene (PTFE), copolymers of polyamide and polyether, or a combination thereof. In some instances, the outer shaft 318 may include a reinforcing element (for example, a laser-cut tube). In certain embodiments, the outer shaft 318 may be injection molded or extruded. In some embodiments, the catheter shaft 304 is coated for lubricity with a hydrophilic coating, or other types of coating suitable for coating a catheter shaft as known by a skilled person in the art.

In certain examples, the outer shaft 318 and/or the catheter shaft 304 may house all of the catheter components until the desired target location is reached. In some embodiments, once the shunting catheter 302 has reached the target location, the outer shaft 318 may translate towards the proximal end of the catheter shaft 304 to expose the shunting element 306 and other components.

In certain embodiments, the apposition element 308 is flexible and compressed to fit between the outer shaft 318 and the catheter shaft 304 during deployment. According to certain embodiments, the apposition element 308 is disposed within the outer shaft 318 at a first apposition state (shown in phantom lines—for example, during deployment). In some embodiments, the apposition element 308 is uncovered by the outer shaft 318 and extends outwardly from the catheter shaft 304 to contact a vessel wall at a second apposition state (shown in solid lines). In certain embodiments, the apposition element 308 can appose to a cardiovascular system wall (for example, the front wall or back wall of the CS, a left atrium wall, a right atrium wall, etc.) at the second apposition state, for example, to help position and/or stabilize the shunting element 306.

In some embodiments and as illustrated, the apposition element 308 includes a first end 320 and a second end 322, and the first end 320 is secured to the catheter shaft 304 and the second end 322 is disposed apart from the catheter shaft 304. In other embodiments, the first end 320 is movable relative to the catheter shaft 304 and the second end 322 is secured to the catheter shaft 304.

In certain embodiments, the apposition element 308 includes a plurality of movable struts 324 that together define an expandable cage 326. In some embodiments, one or more of the movable struts 324 includes a first strut portion 328 and a second strut portion 330, the first strut portion 328 and an axis 332 defined by the catheter shaft 304 form an expansion angle 336 in the second apposition state, and the expansion angle 336 is greater than zero degrees. In certain embodiments, the expansion angle 336 is greater than 15 degrees. In some embodiments, the first strut portion 328 and the second strut portion 330 form a strut angle 334 greater than zero degrees in the second apposition state. In certain embodiments, one or more of the movable struts 324 includes one or more holes 338. In some embodiments, the holes 338 of the struts 324 and/or the spaces between adjacent struts 324 allow blood flow through the apposition element 308, thus reducing the risk of thrombus formation caused by any occlusion in the vessel.

In some embodiments, the apposition element 308 has a first height from the catheter shaft 304 at the first apposition state and a second height from the catheter shaft 304 at the second apposition state, and the second height is greater than the first height. In some embodiments, the second height is at least two times the first height. In certain embodiments, the first height is in a range of 0 mm to 1 mm. In certain embodiments, the second height is in a range of 4 mm to 10 mm.

In some embodiments and as illustrated, the apposition element 308 is disposed substantially symmetrically around the catheter shaft 304, more specifically the struts 324 are disposed at equal angles around the catheter shaft 304. In other embodiments, the apposition element 308 is disposed substantially non-symmetrically around the catheter shaft 304, more specifically the struts 324 are disposed at unequal angles around the catheter shaft 304.

In certain embodiments, the movable struts 324 comprise braided wires. In certain embodiments, the apposition element 308 comprises a laser-cut component. In some embodiments, the struts 324 are self-movable (for example, by being made of a shape memory material and set in the expanded state) and the apposition element 308 is thereby self-expandable. In certain embodiments, the struts are movable via an actuator (for example, an inflatable balloon) carried within the cage. In certain embodiments, the struts 324 are constructed of at least one material selected from a group consisting of nitinol, stainless steel, titanium, platinum-iridium, and cobalt-chromium.

In some embodiments, the shunting catheter 302 has a diameter of from about 2 mm to about 6 mm. In certain embodiments, the shunting catheter 302 has a diameter from about 2.5 mm to about 5 mm. In some embodiments, the shunting catheter 302 has a diameter from about 3 mm to about 4 mm. In certain embodiments, the shunting catheter 302 may have a diameter allowing it to pass through vessels and parts of the cardiovascular system to reach a target location.

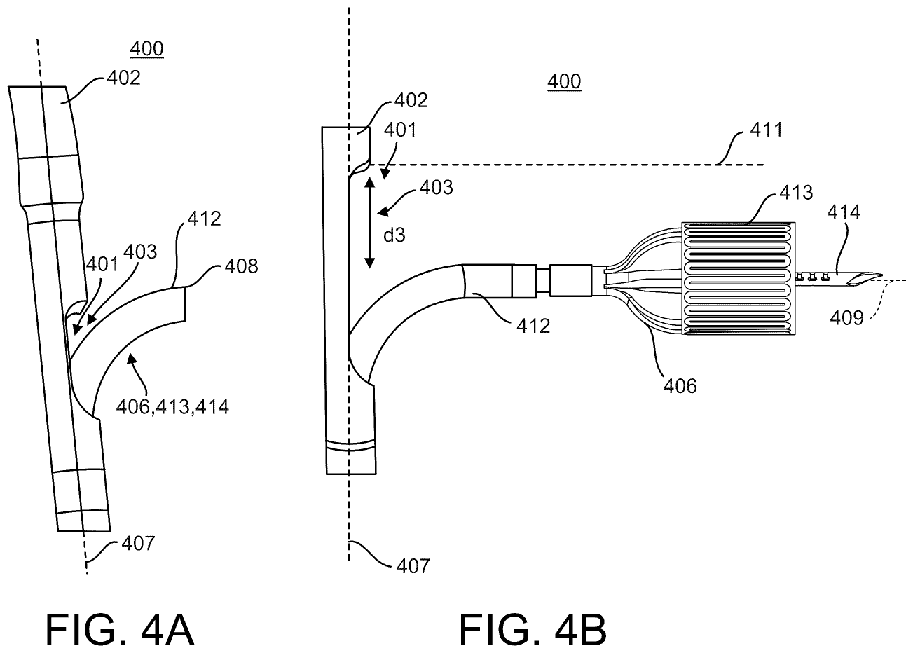

FIGS. 4A-4B are schematic diagrams of side views of an example of a shunting catheter 400, in accordance with embodiments of the present disclosure. In some embodiments and as shown in FIGS. 4A-4B, the shunting catheter 400 includes a catheter shaft 402 having a shaft lumen 401, a shaft opening 403, and a shunting element 406, more specifically an ablation assembly 406, disposed within the shaft lumen 401 at a first shunting element state (for example, during deployment to position the ablation assembly 406, as shown in FIG. 4A), and extended from the catheter shaft 402 at a second shunting element state (for example, during shunting, as shown in FIG. 4B). In some embodiments, the ablation assembly 406 includes a crimping shaft 412 having a predetermined curve for a shunting element 413 to deploy, and a puncture element 414.

According to some embodiments, the ablation assembly 406 may have a telescoping feature (for example, the shunting element 413 and the puncture element 414 being retractable into the crimping shaft 412, as shown in FIG. 4A) to allow the blunt distal end 408 of the crimping shaft 412 to contact the wall between the patient's LA and CS before the puncture element 414 is translated forward to make contact with the wall between the patient's LA and CS. In embodiments, the telescoping feature of the ablation assembly 406 allows for a safe delivery of the puncture element 414 to the target location.

According to certain embodiments, the ablation assembly 406 of the shunting catheter 400 has a first deployment state (for example, shown in FIG. 4A) and a second deployment state (for example, shown in FIG. 4B). In some embodiments, at the first deployed state the shunting element 413 and the puncture element 414 are retracted in a lumen of the crimping shaft 412. In some embodiments, at the second deployed state the shunting element 413 and the puncture element 414 are extended from a distal end 408 of the crimping shaft 412.

In certain embodiments, the shaft opening 403 includes an edge defining an opening axis 411. In some embodiments, the opening axis 411 may be generally perpendicular to a first axis 407 along the catheter shaft 402. In some embodiments, the distance (d3) between the opening axis 411 and a second axis 409 along the ablation assembly 406 may be from about 3 mm to about 20 mm.

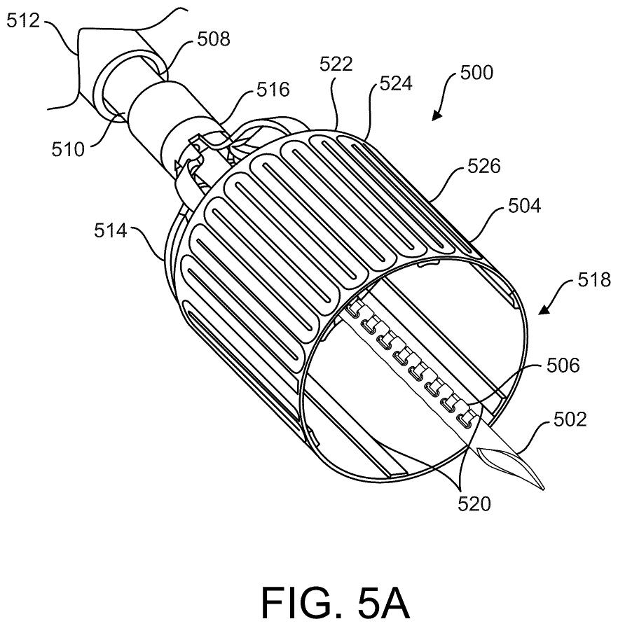

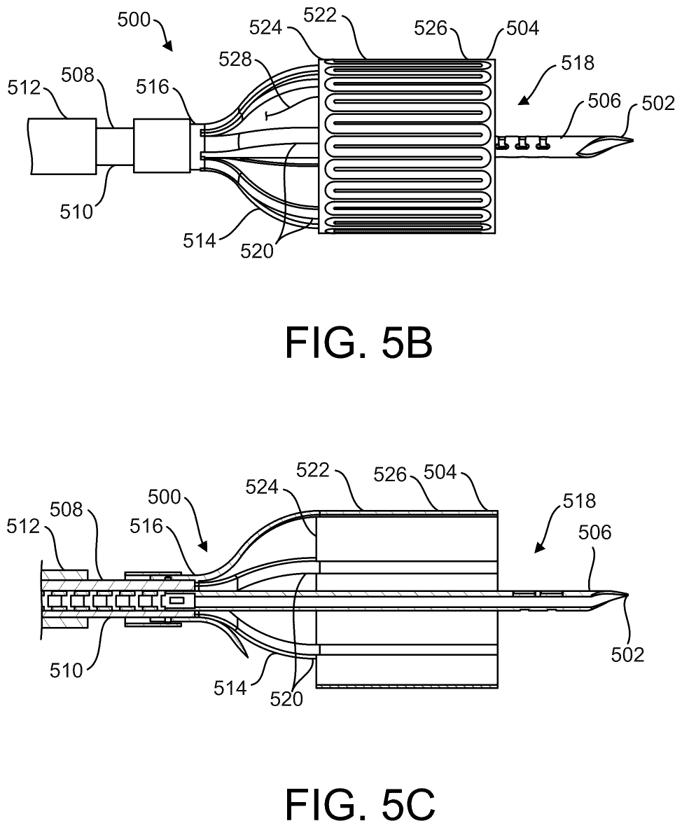

FIGS. 5A-5C are schematic diagrams of views of an example of a shunting element 500, more specifically an expandable ablation assembly 500, in accordance with embodiments of the present disclosure. In certain embodiments, the ablation assembly 500 may include a puncture element 502 and a shunting element 504. In some embodiments, the puncture element 502 is coupled to a distal end of an inner member 506, more specifically a laser-cut tube, of an ablation shaft 508. In some embodiments, the puncture element 502 may be configured to puncture an opening at a target location in a patient, such as a vessel wall, more specifically the wall between the CS and LA of the patient. In certain embodiments, the shunting element 504 has a length in a range of 3 mm to 15 mm. In certain embodiments, the shunting element 504 has an expanded diameter in a range of 2 mm to 10 mm.

According to some embodiments, the puncture element 502 (for example, a needle) may take on many different needle configurations. Configurations for the puncture element 502 may include, but not are not limited to, regular trocar point, regular taper point, regular taper cutting, regular reverse cutting edge, regular diamond point, regular conventual cutting edge, regular blunt taper point, premium lancet point, premium diamond point, and/or premium cutting edge. In certain embodiments, the puncture element 502 is made of materials including nitinol, stainless steel, cobalt chromium, aluminum, and/or a combination thereof. In certain embodiments, the puncture element 502 physically contacts tissue to puncture an opening at the target location in the patient. In certain embodiments, the puncture element 502 receives energy from an energy source and delivers ablation energy (such as radiofrequency (RF) energy, phased RF energy, cryogenic energy, thermal energy, pulse energy, laser energy, ultrasound energy, microwave energy, and/or the like) to tissue to puncture an opening at the target location in the patient.

The shunting element 504 is disposed on and/or within an outer member 510 of the ablation shaft 508, and the shunting element 504 and the ablation shaft 508 are together slidable into and out of a lumen of a crimping shaft 512. In certain embodiments, after the puncture element 502 forms an opening in the tissue at a target location in a patient, the shunting element 504 expands to enlarge the opening in the tissue. In some embodiments, the shunting element 504 then receives energy from an energy source and delivers ablation energy (such as radiofrequency (RF) energy, phased RF energy, cryogenic energy, thermal energy, pulse energy, laser energy, ultrasound energy, microwave energy, and/or the like) to ablate the tissue and thereby solidify the opening at the target location.

In some embodiments, the shunting element 504 includes an expandable cage 514, and the expandable cage 514 is fixedly coupled at its proximal end 516 to the ablation shaft 508 (for example, via reflow soldering or adhesives). In certain embodiments, the expandable cage 514 illustratively includes an open distal end 518 (that is, being disposed apart from the inner member 506 of the ablation shaft 508). Additionally, or alternatively, the expandable cage 514 may taper toward the distal end 518 and movably couple to the inner member 506 of the ablation shaft 508 at the distal end 518. As another example, the expandable cage 514 may taper toward the distal end 518, fixedly couple to the inner member 506 of the ablation shaft 508 at the distal end 518, and movably couple to the inner member 506 of the ablation shaft 508 at the proximal end 516.

In certain embodiments, the expandable cage 514 is made of one or more materials that facilitate self-expansion, such as nitinol, or one or more other materials, such as stainless steel, titanium, platinum-iridium, or cobalt-chromium.

In certain embodiments and as illustrated, the expandable cage 514 includes a plurality of movable struts 520. In some embodiments, the struts 520 are collapsed radially inwardly, or toward each other, when the shunting element 504 is disposed in the crimping shaft 512 (that is, in a first state; not specifically illustrated). In certain embodiments, the struts 520 move radially outwardly, or away from each other, when the shunting element is disposed outside of the crimping shaft 512 (that is, in a second state, as illustrated in FIGS. 5A-5C). In certain embodiments and as illustrated, the expandable cage 514 includes six movable struts 520. In other embodiments, the expandable cage 514 includes a different number of movable struts 520, such as two, three, four, five, seven, eight, nine, ten, or more movable struts 520. In certain embodiments and as illustrated, the struts 520 are self-movable (for example, by being constructed of a shape memory material and set in the expanded state). In certain embodiments, the self-movement of the struts 520 may be controlled by a constrainer, such as a plurality of sutures (for example, thin strands) coupled to the struts 520. Such a constrainer may be reconfigured from a constraining state to a release state to permit the struts 520 to move, and the constrainer may be reconfigured from the release state to the constraining state to collapse the struts 520. In some embodiments, the constrainer may be coupled to distal ends of the struts. In certain embodiments, the struts 520 are expanded by an actuator, such as an inflatable balloon (not shown). In certain embodiments, an actuator may be disposed in a cavity of the cage 514 formed between the struts 520. In certain embodiments, one or more additional components, such as control wires coupled to the actuators, may be disposed in the cavity of the cage 514 and extend through the ablation shaft 508. In certain embodiments, such as those in which the distal end 518 of the expandable cage 514 fixedly couple to the inner member 506 of the ablation shaft 508 and the proximal end 516 movably couples to the inner member 506 of the ablation shaft 508, the actuator may be a slidable intermediate shaft (not shown).

In certain embodiments and as illustrated, the expandable cage 514 supports and/or carries an electrode structure 522 including one or more electrodes. In some embodiments, the electrode structure 522 may include a thin film electrode. In some embodiments, the electrode structure 522 includes a thin film substrate and one or more electrodes are disposed on the thin film substrate. In certain embodiments, the one or more electrodes includes two or more electrodes. In some embodiments, at least two of the one or more electrodes form an electrode pair. In certain embodiments, the electrode structure 522 has a length in a range of 4 mm to 12 mm. In certain embodiments, the electrode structure 522 has an expanded diameter in a range of 2 mm to 10 mm. In certain embodiments, the electrode structure 522 includes a base 524 that supports and/or carries one or more conductors 526. In some embodiments, the base 524 is made of a flexible/foldable material (such as an insulating polymer, more specifically a polyimide) and thereby moves with the expandable cage 514 from the first state to the second state and vice versa.

In some embodiments and as illustrated, the conductor 526 is carried on an outer surface of the base 524, and the conductor 526 is configured to contact tissue at a target location in a patient and deliver ablation energy to the tissue. In certain embodiments, the conductor 526 is made of one or more conductive metals, such as gold, platinum-iridium, copper, or the like. In certain embodiments and as illustrated, the conductor 526 includes a serpentine shape, more specifically, the conductor 526 includes longitudinally extending segments joined by short turns near the longitudinal ends of the base 524. In certain embodiments, the conductor 526 may have different shapes, although conductors 526 having serpentine shapes may experience lower strain upon expansion and contraction relative to other shapes.

In certain embodiments, one or more lead wires 528 (FIG. 5B) couple the conductor 526 to an energy source. In some embodiments, the lead wires 528 may extend through one of the shafts (for example, the crimping shaft 512 or the ablation shaft 508) or outside of the shafts.

In some embodiments, the expandable cage 514 supports and/or carries a plurality of electrodes 522. In certain embodiments, the plurality of electrodes 522 are disposed on a surface of the expandable cage 514. In some embodiments, the plurality of electrodes 522 are disposed on an outer surface of the expandable cage. In some embodiments, the shunting element 504 lacks the electrode structure 522, and the movable struts 520 instead contact tissue at a target location within a patient and act as electrodes to deliver ablation energy to the tissue. In such embodiments, one or more portions of the movable struts 520 may carry an insulating coating (such as polyether ether ketone (PEEK), expanded polytetrafluoroethylene (ePTFE), elastomeric coatings, or the like) to inhibit delivery of ablation energy to the blood of the patient.

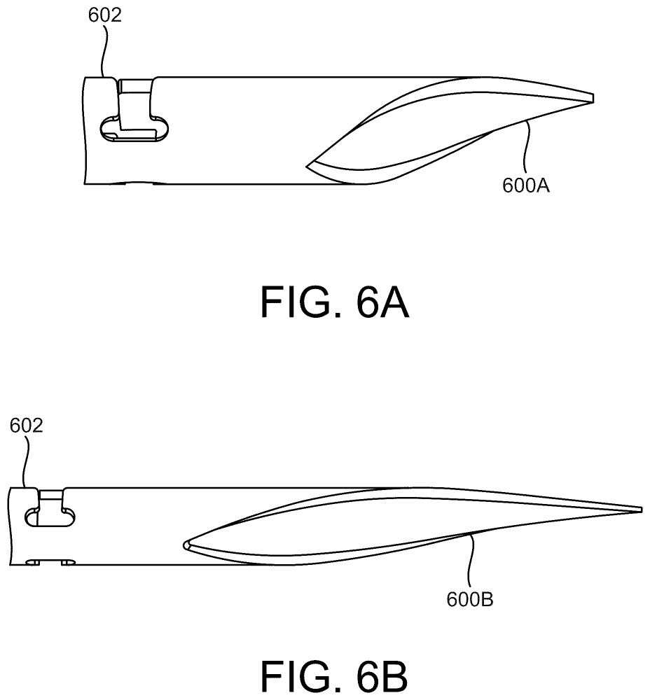

FIGS. 6A-6B are schematic diagrams of side views of examples of puncture elements 600A and 600B of ablation assemblies and inner members 602 of ablation shafts, in accordance with embodiments of the present disclosure. In certain embodiments, the puncture elements 600A and 600B and the inner members 602 are made of materials including nitinol, stainless steel, cobalt chromium, aluminum, and/or a combination thereof. In some embodiments, the puncture elements 600A and 600B and the inner members 602 are hollow needles. More specifically, in some embodiments the puncture elements 600A and 600B and the inner members 602 are hollow to facilitate delivering guidewires therethrough. In some embodiments, the puncture elements 600A and 600B may be coupled to the inner members 602 of the ablation shafts in various manners, such as via laser welding. In certain embodiments, the puncture elements 600A and 600B and the inner members 602 of the ablation shafts may have outer diameters in a range of 0.02 in. to 0.08 in., more specifically about 0.05 in. In some embodiments, the puncture elements 600A and 600B may be coupled to pull wires (not shown) to facilitate bending and steering of the inner members 602 of the ablation shafts.

According to some embodiments, for example as shown in FIG. 6A, the puncture element 600A has a relatively short needle tip shape. In certain embodiments, the puncture element 600A has a tip length of about 0.175 in. (from the end of the tip to a pull wire interface—not shown). According to some embodiments, for example as shown in FIG. 6B, the puncture element 600B has a relatively long needle tip shape. In certain embodiments, the puncture element 600B has a tip length of about 0.325 in. (from the end of the tip to a pull wire interface—not shown).

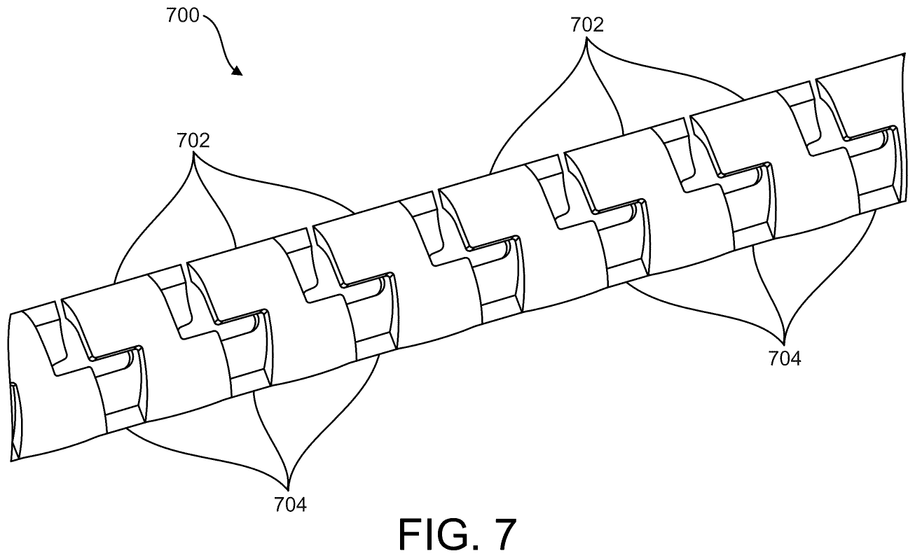

FIG. 7 is a schematic diagram of a perspective view of an example of an inner member 700 of an ablation shaft, in accordance with embodiments of the present disclosure. In certain embodiments, the inner member 700 is made of materials including nitinol, stainless steel, cobalt chromium, aluminum, and/or a combination thereof. In certain embodiments, the inner member 700 is bendable and steerable via one or more pull wires (not shown). In some embodiments, to facilitate such bending and steering, the inner member 700 may be hollow and include a plurality of ribs 702 separated by voids 704. In some embodiments, the inner member 700 may have a different structure.

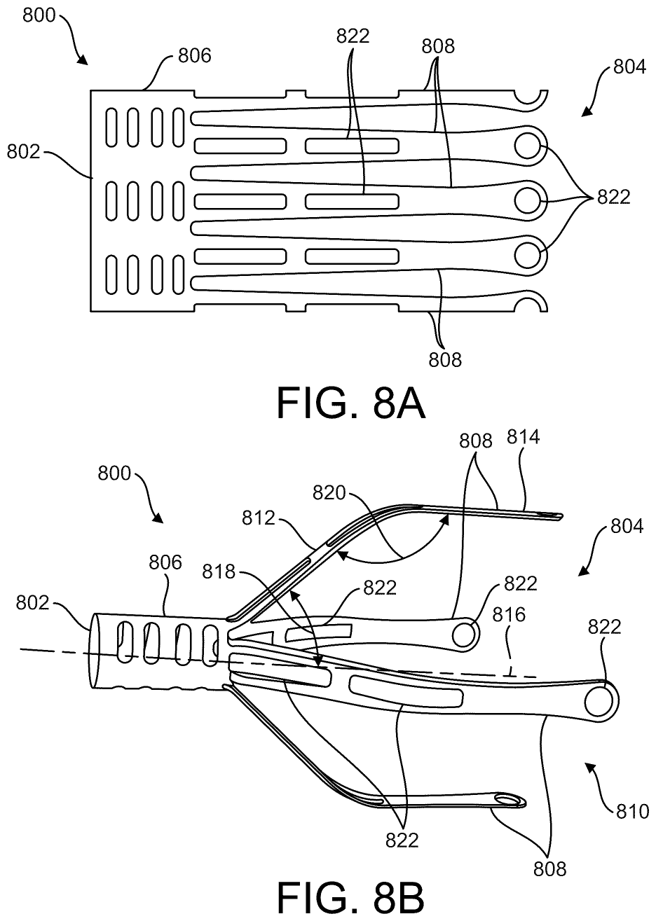

FIGS. 8A-8B are schematic diagrams of views of an example of an apposition element 800 of a shunting catheter, in accordance with embodiments of the present disclosure. FIG. 8A illustrates the apposition element 800 in a flat initial configuration (for example, a configuration of the apposition element 800 during manufacturing thereof and before coupling the apposition element 800 to a catheter shaft of a shunting catheter). FIG. 8B illustrates the apposition element 800 in a shaped configuration (for example, a configuration of the apposition element 800 after manufacturing thereof and before coupling the apposition element 800 to a catheter shaft of a shunting catheter).

In certain embodiments, the apposition element 800 is flexible and compressed to fit between an outer shaft and a catheter shaft of a shunting catheter (shown elsewhere) during deployment. According to certain embodiments, the apposition element 800 is disposed within the outer shaft at a first apposition state (not shown—for example, during deployment). In some embodiments, the apposition element 800 is uncovered by the outer shaft and extends outwardly from the catheter shaft at a second apposition state (for example, as shown in FIG. 8B). In certain embodiments, the apposition element 800 can appose to a cardiovascular system wall (for example, the front wall or back wall of the CS, a left atrium wall, a right atrium wall, etc.) at the second apposition state, for example, to help position and/or stabilize a shunting element of the shunting catheter.

In some embodiments and as illustrated, the apposition element 800 includes a first end 802 and a second end 804, and the first end 802 is secured to the catheter shaft and the second end 804 is disposed apart from the catheter shaft. In other embodiments, the first end 802 is movable relative to the catheter shaft and the second end 804 is secured to the catheter shaft.

In certain embodiments, the apposition element 800 includes a base 806 that defines the first end 802 and a plurality of movable struts 808, illustratively four struts 808, that define the second end 804. In some embodiments, the apposition element 800 has a different number of struts 808, such as two, three, five, six, seven, eight, nine, ten, or more struts 808. Referring specifically to FIG. 8B, in some embodiments the struts 808 extend from the base 806 and together define an expandable cage 810. In some embodiments one or more of the movable struts 808 includes a first strut portion 812 and a second strut portion 814, the first strut portion 812 and a longitudinal axis 816 defined by the base 806 form an expansion angle 818 in the second apposition state, and the expansion angle 818 is greater than zero degrees. In certain embodiments, the expansion angle 818 is greater than 15 degrees. In some embodiments, the first strut portion 812 and the second strut portion 814 form a strut angle 820 greater than zero degrees in the second apposition state. In certain embodiments, one or more of the movable struts 808 includes one or more holes 822. In certain embodiments, one or more of the holes 822 are elongated in a direction parallel to the longitudinal axis 816 of the apposition element 800. In some embodiments, the holes 822 of the struts 808 and/or the spaces between adjacent struts 808 allow blood flow through the apposition element 800, thus reducing the risk of thrombus formation caused by any occlusion in the vessel.

In some embodiments, the apposition element 800 has a first height from the catheter shaft at the first apposition state and a second height from the catheter shaft at the second apposition state, and the second height is greater than the first height. In some embodiments, the second height is at least two times the first height. In certain embodiments, the first height is in a range of 0 mm to 1 mm. In certain embodiments, the second height is in a range of 4 mm to 10 mm.

In some embodiments and as illustrated, the struts 808 are disposed substantially symmetrically around the base 806 (and, as a result, the catheter shaft), more specifically the struts 808 are disposed at equal angles around the base 806. In other embodiments, the struts 808 are disposed substantially non-symmetrically around the base 806 (and, as a result, the catheter shaft), more specifically the struts 808 are disposed at unequal angles around the base 806.

In certain embodiments, the apposition element 800 comprises a laser-cut component. In some embodiments, the struts 808 are self-movable (for example, by being made of a shape memory material and set in the expanded state) and the apposition element 800 is thereby self-expandable. In certain embodiments, the struts 808 are movable via an actuator (for example, an inflatable balloon) carried within the cage 810. In certain embodiments, the struts 808 are constructed of at least one material selected from a group consisting of nitinol, stainless steel, titanium, platinum-iridium, and cobalt-chromium.

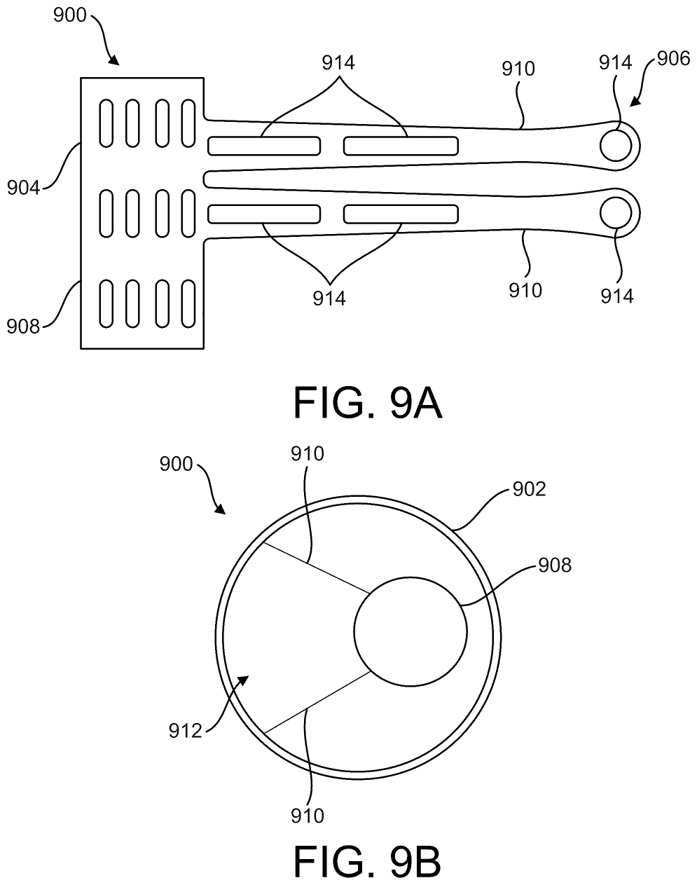

FIGS. 9A-9B are schematic diagrams of views of an example of an apposition element 900, in accordance with embodiments of the present disclosure. FIG. 9A is a side view of the apposition element 900 in a flat initial configuration (for example, a configuration of the apposition element 900 during manufacturing thereof and before coupling the apposition element 900 to a catheter shaft of a shunting catheter). FIG. 9B is an end view of the apposition element 900 in a shaped configuration (for example, a configuration of the apposition element 900 after manufacturing thereof and after coupling the apposition element 900 to a catheter shaft of a shunting catheter) and apposing to a vessel wall 902 of a patient (for example, the front wall or back wall of the CS, a left atrium wall, a right atrium wall, etc.).

In certain embodiments, the apposition element 900 is flexible and compressed to fit between an outer shaft and a catheter shaft of a shunting catheter (shown elsewhere) during deployment. According to certain embodiments, the apposition element 900 is disposed within the outer shaft at a first apposition state (not shown—for example, during deployment). In some embodiments, the apposition element 900 is uncovered by the outer shaft and extends outwardly from the catheter shaft at a second apposition state (for example, as shown in FIG. 9B). In certain embodiments, the apposition element 900 can appose to the vessel wall 902 at the second apposition state, for example, to help position and/or stabilize a shunting element of the shunting catheter.

In some embodiments and as illustrated, the apposition element 900 includes a first end 904 and a second end 906, and the first end 904 is secured to the catheter shaft and the second end 906 is disposed apart from the catheter shaft. In other embodiments, the first end 904 is movable relative to the catheter shaft and the second end 906 is secured to the catheter shaft.

In certain embodiments, the apposition element 900 includes a base 908 that define the first end 904 and a plurality of movable struts 910, illustratively two struts 910, that define the second end 906. In some embodiments, the apposition element 900 has a different number of struts 910, such as three, four, five, six, seven, eight, nine, ten, or more struts 910. Referring specifically to FIG. 9B, in some embodiments the struts 910 extend from the base 908 and together define an expandable cage 912. In certain embodiments, one or more of the movable struts 910 includes one or more holes 914. In some embodiments, the holes 914 of the struts 910 and/or the spaces between adjacent struts 910 allow blood flow through the apposition element 900, thus reducing the risk of thrombus formation caused by any occlusion in the vessel.

In some embodiments, the apposition element 900 has a first height from the catheter shaft at the first apposition state and a second height from the catheter shaft at the second apposition state, and the second height is greater than the first height. In some embodiments, the second height is at least two times the first height. In certain embodiments, the first height is in a range of 0 mm to 1 mm. In certain embodiments, the second height is in a range of 4 mm to 20 mm.

In some embodiments and as illustrated, the struts 910 are disposed substantially non-symmetrically around the base 908 (and, as a result, the catheter shaft), more specifically the struts 910 are disposed at unequal angles around the base 908. In other embodiments, the struts 910 are disposed substantially symmetrically around the base 908 (and, as a result, the catheter shaft), more specifically the struts 910 are disposed at equal angles around the base 908.

In certain embodiments, the apposition element 900 comprises a laser-cut component. In some embodiments, the struts 910 are self-movable (for example, by being made of a shape memory material and set in the expanded state) and the apposition element 900 is thereby self-expandable. In certain embodiments, the struts 910 are movable via an actuator (for example, an inflatable balloon) carried within the cage 912. In certain embodiments, the struts 910 are constructed of at least one material selected from a group consisting of nitinol, stainless steel, titanium, platinum-iridium, and cobalt-chromium.

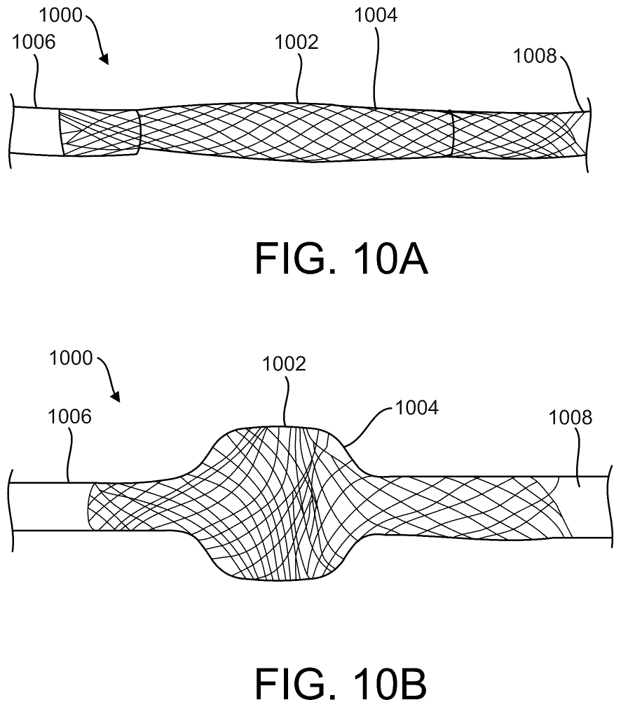

FIGS. 10A-10B are schematic diagrams of views of another example of an apposition element 1000 of a shunting catheter, in accordance with embodiments of the present disclosure. FIG. 10A illustrates the apposition element 1000 in a first apposition state, and FIG. 10B illustrates the apposition element 1000 in a second apposition state. According to certain embodiments, the apposition element 1000 is disposed within an outer shaft of a shunting catheter (shown elsewhere) at the first apposition state. In some embodiments, the apposition element 1000 is uncovered by the outer shaft and extends outwardly from a catheter shaft of the shunting catheter (shown elsewhere) at the second apposition state. In certain embodiments, the apposition element 1000 can appose to a wall of the vessel (for example, the front wall or back wall of the CS, a left atrium wall, a right atrium wall, etc.) at the second apposition state, for example, to help position and/or stabilize a shunting element of the shunting catheter.

In certain embodiments, the apposition element 1000 is made of one or more materials that facilitate self-expansion, such as nitinol, or one or more other materials, such as stainless steel, titanium, platinum-iridium, or cobalt-chromium.

The apposition element 1000 includes a plurality of movable struts, more specifically a plurality of braided wires 1002. In some embodiments, the wires 1002 together define an expandable cage 1004. In certain embodiments and as illustrated, the braided wires 1002 may generally include a first set of wires that extend helically in a first direction and a second set of wires that extend helically in a second, opposite direction. Alternatively, the braided wires 1002 may have different arrangements. In some embodiments, the spaces between adjacent wires 1002 allow blood flow through the apposition element 1000, thus reducing the risk of thrombus formation caused by any occlusion in the vessel.

In some embodiments and as illustrated, the apposition element 1000 includes a first end 1006 and a second end 1008, and the first end 1006 is secured to the catheter shaft and the second end 1008 is movable relative to the catheter shaft. In other embodiments, the first end 1006 is secured to the catheter shaft and the second end 1008 is disposed apart from the catheter shaft.

In some embodiments, the apposition element 1000 has a first height from the catheter shaft at the first apposition state and a second height from the catheter shaft at the second apposition state, and the second height is greater than the first height. In some embodiments, the second height is at least two times the first height. In certain embodiments, the first height is in a range of 0 mm to 1 mm. In certain embodiments, the second height is in a range of 4 mm to 10 mm.

In some embodiments and as illustrated, the wires 1002 are disposed substantially symmetrically around the catheter shaft. In other embodiments, the wires 1002 are disposed substantially non-symmetrically around the catheter shaft.

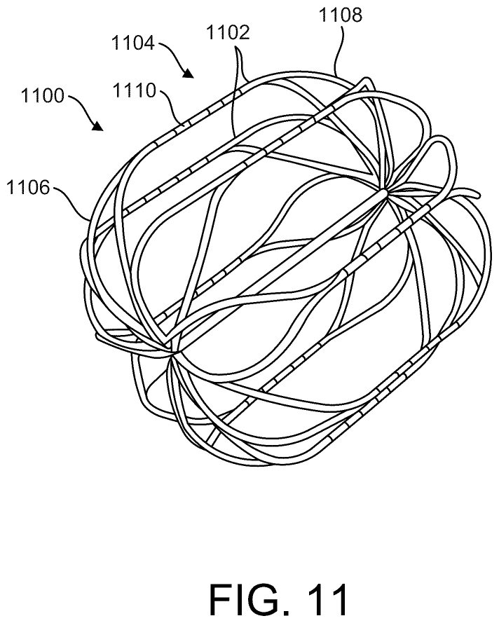

FIG. 11 is a schematic diagram of a perspective view of another example of an apposition element 1100 of a shunting catheter, in accordance with embodiments of the present disclosure. In certain embodiments, the apposition element 1100 is flexible and compressed to fit between an outer shaft and a catheter shaft of a shunting catheter (shown elsewhere) during deployment. According to certain embodiments, the apposition element 1100 is disposed within the outer shaft at a first apposition state (not shown—for example, during deployment). In some embodiments, the apposition element 1100 is uncovered by the outer shaft and extends outwardly from the catheter shaft at a second apposition state (for example, as shown in FIG. 11). In certain embodiments, the apposition element 1100 can appose to a cardiovascular system wall (for example, the front wall or back wall of the CS, a left atrium wall, a right atrium wall, etc.) at the second apposition state, for example, to help position and/or stabilize a shunting element of the shunting catheter.

According to certain embodiments, the apposition element 1100 includes a plurality of movable struts 1102, illustratively eight struts 1102, that together define an expandable cage 1104. In some embodiments, the apposition element 1100 has a different number of struts 1102, such as two, three, four, five, six, seven, nine, ten, or more struts 1102. In certain embodiments, each movable strut 1102 includes a proximal loop 1106, a distal loop 1108, and a longitudinally extending portion 1110 coupling the loops 1106 and 1108. In other embodiments, the movable struts 1102 have different structures. In some embodiments, the spaces between adjacent struts 1102 allow blood flow through the apposition element 1100, thus reducing the risk of thrombus formation caused by any occlusion in the vessel.

In some embodiments, the apposition element 1100 has a first height from the catheter shaft at the first apposition state and a second height from the catheter shaft at the second apposition state, and the second height is greater than the first height. In some embodiments, the second height is at least two times the first height. In certain embodiments, the first height is in a range of 0 mm to 1 mm. In certain embodiments, the second height is in a range of 4 mm to 10 mm.

In some embodiments and as illustrated, the struts 1102 are disposed substantially symmetrically around the catheter shaft, more specifically the struts 1102 are disposed at equal angles around the catheter shaft. In other embodiments, the struts 1102 are disposed substantially non-symmetrically around the catheter shaft, more specifically the struts 1102 are disposed at unequal angles around the catheter shaft.

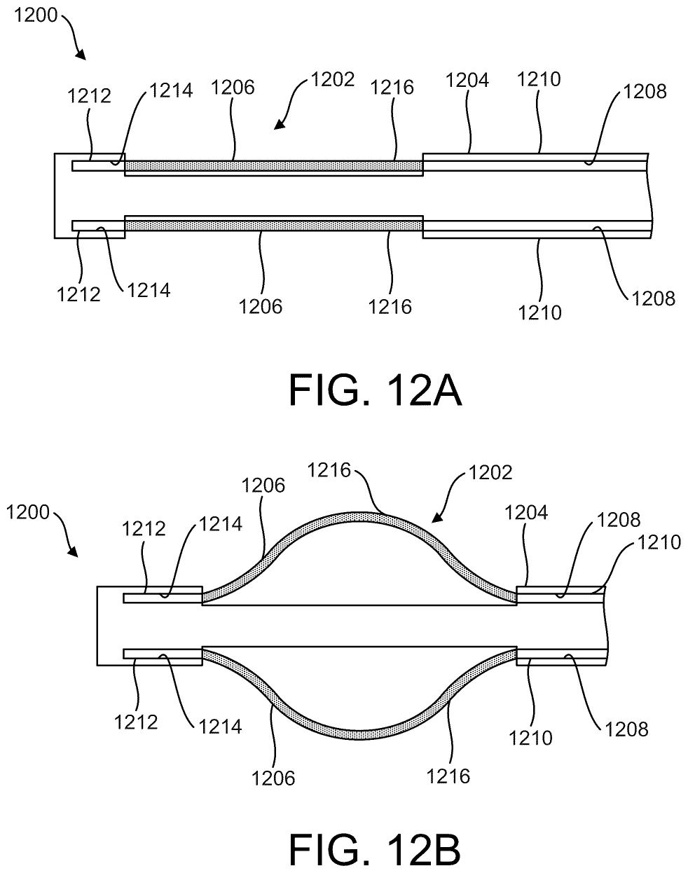

FIGS. 12A-12B are schematic diagrams of views of another example of a shunting catheter 1200, in accordance with embodiments of the present disclosure. FIG. 12A illustrates an apposition element 1202 of the shunting catheter 1200 in a first apposition state, and FIG. 12B illustrates the apposition element 1202 in a second apposition state. According to certain embodiments, the apposition element 1202 is retracted against a catheter shaft 1204 of the shunting catheter 1200 at the first apposition state. In some embodiments, the apposition element 1202 extends outwardly from the catheter shaft 1204 at the second apposition state. In certain embodiments, the apposition element 1202 can appose to a wall of the vessel (for example, the front wall or back wall of the CS, a left atrium wall, a right atrium wall, etc.) at the second apposition state, for example, to help position and/or stabilize a shunting element of the shunting catheter.

The apposition element 1202 includes a plurality of movable struts 1206, which may be a plurality of wires. In some embodiments, the struts 1206 define an expandable cage. In some embodiments, the spaces between adjacent struts 1206 allow blood flow through the apposition element 1202 in the second apposition state, thus reducing the risk of thrombus formation caused by any occlusion in the vessel.

In certain embodiments, the catheter shaft 1204 includes one or more apposition element lumens 1208, and each lumen 1208 includes and/or carries one of the struts 1206 of the apposition element 1202. In some embodiments, a proximal end portion 1210 of each strut 1206 is movable in one of the lumens 1208, and a distal end portion 1212 of each strut 1206 is inhibited from moving distally relative to the catheter shaft 1204. In certain embodiments, a distal end portion 1212 is secured to the catheter shaft 1204 within a distal lumen 1214, for example, via an adhesive. In some embodiments, a distal end portion 1212 is not secured to the catheter shaft 1204, although the distal end portions 1212 contact the catheter shaft 1204 within the distal lumens 1214 such that distal movement of the distal end portions 1212 of the struts 1206 relative to the catheter shaft 1204 is inhibited. In certain embodiments and as illustrated, intermediate portions 1216 of the struts 1206 are exposed to the exterior of the shunting catheter 1200 between the apposition element lumens 1208 and the distal lumens 1214. In certain embodiments and as shown in FIG. 12B, moving the proximal end portions 1210 of the struts 1206 distally relative to the catheter shaft 1204 causes the intermediate portions 1216 of the struts 1206 to bow and extend outwardly from the catheter shaft 1204 at the second apposition state.

In certain embodiments and as illustrated, the apposition element 1202 includes two struts 1206. In other embodiments, the apposition element 1202 includes a different number of struts 1206, such as one, three, four, five, six, seven, eight, nine, ten or more struts 1206. In some embodiments, the struts 1206 have circular cross-sectional shapes. In some embodiments, the struts 1206 have non-circular cross-sectional shapes, such as rectangular cross-sectional shapes. In some embodiments, the struts 1206 have uniform cross-sectional shapes along their length. In certain embodiments, the struts 1206 have non-uniform cross-sectional shapes along their length. In some embodiments, the proximal end portions 1210 of the struts 1206 have relatively large cross-sectional areas to facilitate pushability, and the intermediate portions 1216 of the struts 1206 have relatively small cross-sectional areas to facilitate flexibility.

In some embodiments, the apposition element 1202 has a first height from the outer surface of the catheter shaft at the first apposition state and a second height from the outer surface of the catheter shaft at the second apposition state, and the second height is greater than the first height. In some embodiments, the second height is at least two times the first height. In certain embodiments, the first height is in a range of −2 mm to 1 mm. In certain embodiments, the second height is in a range of 5 mm to 25 mm. In certain embodiments, each strut 1206 of the apposition element 1202 has the same first height from the catheter shaft 1204 at the first apposition state and the same second height from the catheter shaft 1204 at the second apposition state.

In some embodiments, one or more struts 1206 of the apposition element 1202 has a different first height from the outer surface of the catheter shaft 1204 at the first apposition state compared to one or more other struts 1206 of the apposition element 1202, and/or one or more struts 1206 of the apposition element 1202 has a different second height from the outer surface of the catheter shaft 1204 at the second apposition state compared to one or more other struts 1206 of the apposition element 1202. In some embodiments, the second height of the apposition element 1202 may be user-selectable. For example, in certain embodiments, a user may move the proximal end portions 1210 of the struts 1206 by certain amounts relative to the catheter shaft 1204 to cause the apposition element 1202 to occupy certain second heights. In some embodiments, the shunting catheter 1200 may include an indicator (not shown) that associates certain amounts of movement of the proximal end portions 1210 of the struts 1206 with certain second heights of the apposition element 1202. In certain embodiments, moving the struts 1206 to the second apposition state and/or into contact with a vessel wall may provide haptic feedback to a user.

In some embodiments and as illustrated, the struts 1206 are disposed substantially symmetrically relative to the catheter shaft 1204. In other embodiments, the struts 1206 are disposed substantially non-symmetrically relative to the catheter shaft 1204. In certain embodiments, the struts 1206 are made of one or more materials that facilitate extension away from the catheter shaft 1204, such as a shape memory material set in the second apposition state. In some embodiments, the struts 1206 are made of nitinol, or one or more other materials, such as stainless steel, titanium, platinum-iridium, or cobalt-chromium.

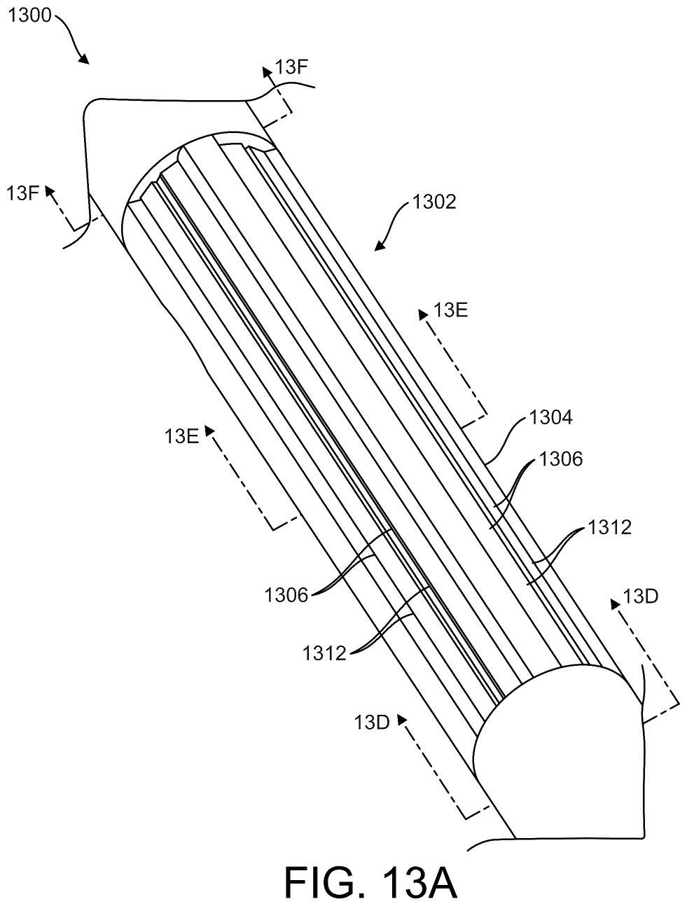

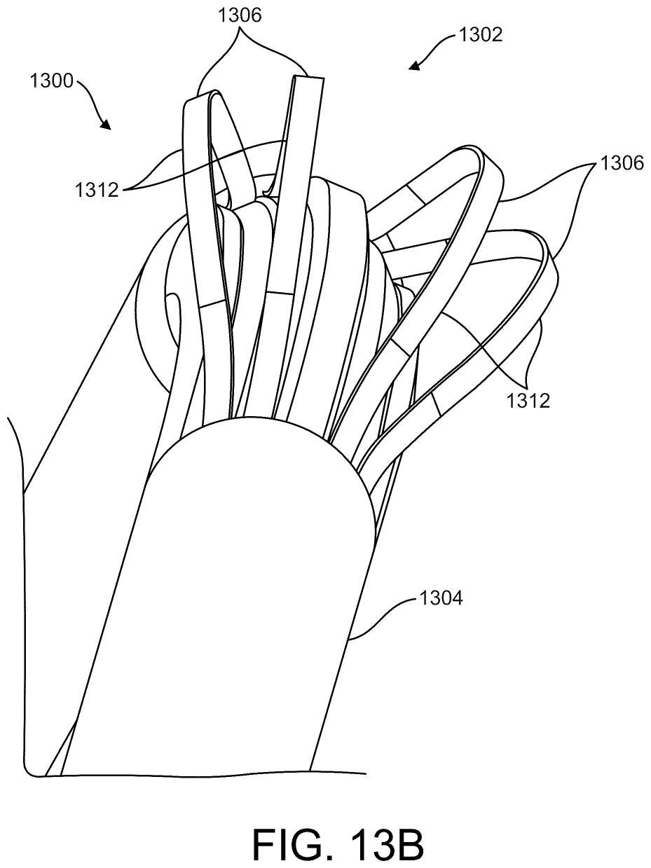

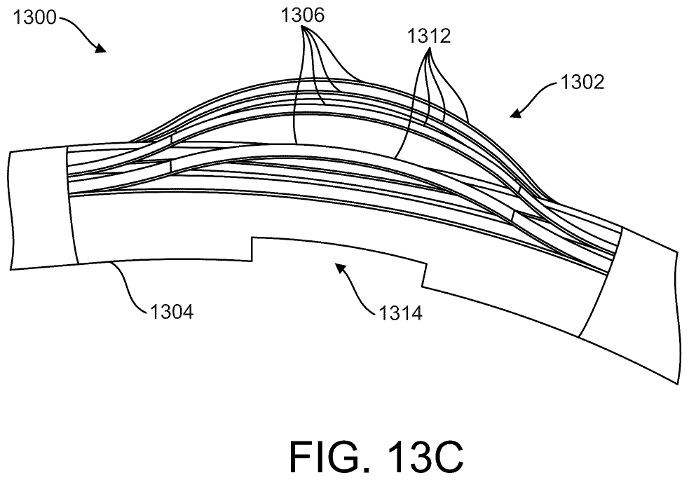

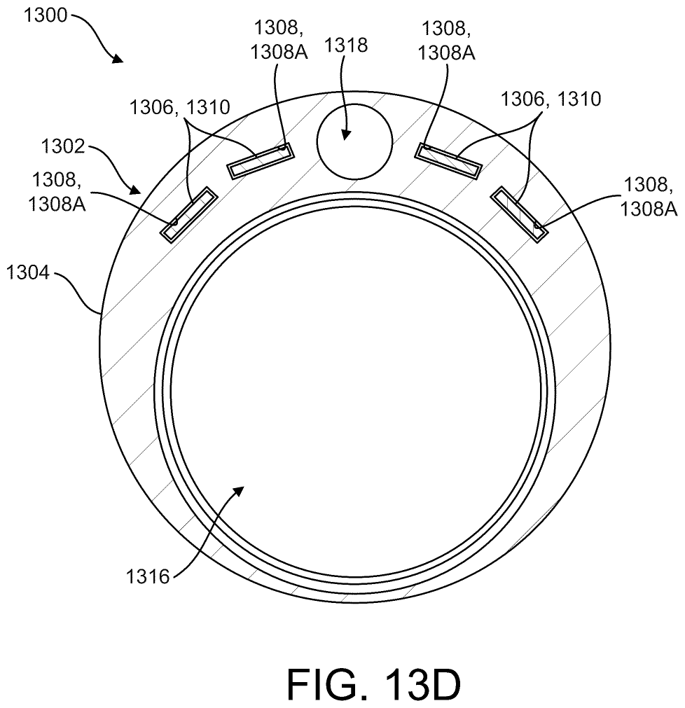

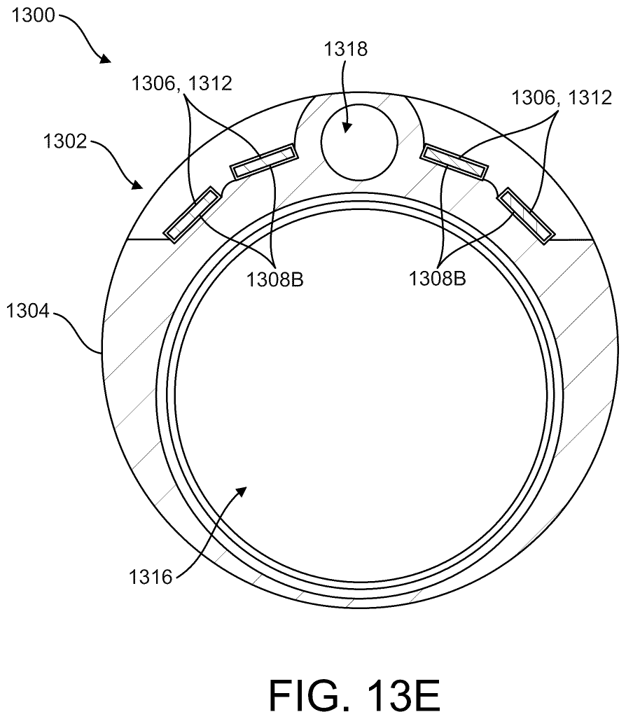

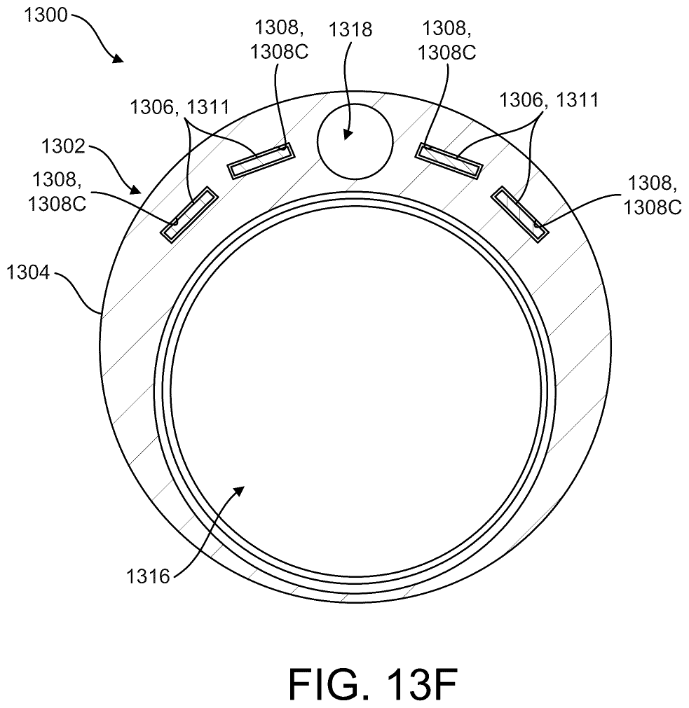

FIGS. 13A-13C are schematic diagrams of views of another example of a shunting catheter 1300, in accordance with embodiments of the present disclosure. FIG. 13A illustrates an apposition element 1302 of the shunting catheter 1300 in a first apposition state, and FIGS. 13B and 13C illustrate the apposition element 1302 in a second apposition state. FIGS. 13D-13F illustrate cross-sectional views of the apposition element 1302 and a catheter shaft 1304 of the shunting catheter 1300 in the first apposition state. According to certain embodiments, the apposition element 1302 is retracted against the catheter shaft 1304 at the first apposition state. In some embodiments, the apposition element 1302 extends outwardly from the catheter shaft 1304 at the second apposition state. In certain embodiments, the apposition element 1302 can appose to a wall of the vessel (for example, the front wall or back wall of the CS, a left atrium wall, a right atrium wall, etc.) at the second apposition state, for example, to help position and/or stabilize a shunting element (not shown) of the shunting catheter 1300.

According to certain embodiments, the apposition element 1302 includes a plurality of movable struts 1306, which may be a plurality of wires. In some embodiments, the struts 1306 together define an expandable cage. In some embodiments, the spaces between adjacent struts 1306 allow blood flow through the apposition element 1302 in the second apposition state, thus reducing the risk of thrombus formation caused by any occlusion in the vessel.

In certain embodiments and as shown specifically in FIG. 13D, the catheter shaft 1304 includes one or more apposition element lumens 1308, and a lumen 1308 includes and/or carries one of the struts 1306 of the apposition element 1302. In some embodiments, a lumen 1308 includes a first lumen section 1308A (FIG. 13D), a second lumen section 1308B (FIG. 13E), and a third lumen section 1308C (FIG. 13F). In certain embodiments, the second lumen section 1308B is between the first lumen section 1308A and the third lumen section 1308C. In some embodiments, the third lumen section 1308C is disposed at or proximate to a distal end of the shunting catheter 1300. In certain embodiments, the first lumen section 1308A and/or the third lumen section 1308C are enclosed structure. In certain embodiments, a cross sectional shape of the first lumen section 1308A (e.g., as illustrated in FIG. 13D) and/or the third lumen section 1308C (e.g., as illustrated in FIG. 13F) is a close shape. In some embodiments, the second lumen section 1308B (e.g., as illustrated in FIG. 13E) is an open structure. In certain embodiments, a cross sectional shape of the second lumen section 1308B is not a closed shape.

In some embodiments, a proximal end portion 1310 (FIG. 13D) of each strut 1306 is movable in one of the lumens 1308, and a distal end portion 1311 (FIG. 13F) of each strut 1306 is inhibited from moving distally relative to the catheter shaft 1304. In certain embodiments, a distal end portion 1311 is secured to the catheter shaft 1304 within the third lumen section 1308C, for example, via an adhesive. In some embodiments, a distal end portion 1311 is not secured to the catheter shaft 1304, although the distal end portions 1311 contact the catheter shaft 1304 within the third lumen section 1308C such that distal movement of the distal end portions 1311 of the struts 1306 relative to the catheter shaft 1304 is inhibited. In certain embodiments and as illustrated, intermediate portions 1312 (FIG. 13E) of the struts 1306 are exposed to the exterior of the shunting catheter 1300 in the second lumen section 1308B. In certain embodiments and as shown in FIG. 13B, moving the proximal end portions 1310 of the struts 1306 distally relative to the catheter shaft 1304 causes the intermediate portions 1312 of the struts 1306 to bow and extend outwardly from the catheter shaft 1304 at the second apposition state.

In certain embodiments and as illustrated specifically in FIG. 13C, the apposition element 1302 is longitudinally aligned with a shaft opening 1314 in the catheter shaft 1304 through which the shunting element is configured to extend. In other embodiments, the apposition element 1302 is longitudinally offset from the opening 1314 in the catheter shaft 1304, for example, by up to 20 mm.

In certain embodiments and as illustrated, the apposition element 1302 includes four struts 1306. In other embodiments, the apposition element 1302 includes a different number of struts 1306, such as one, two, three, five, six, seven, eight, nine, ten or more struts 1306. In some embodiments, the struts 1306 have non-circular cross-sectional shapes, for example and as illustrated, rectangular cross-sectional shapes. In some embodiments, struts 1306 with non-circular cross-sectional shapes have preferential bowing axes (for example, parallel to the long sides of a rectangular cross-sectional shape) to guide movement of the struts 1306 from the first apposition state to the second apposition state. In some embodiments, the struts 1306 have circular cross-sectional shapes. In some embodiments, the struts 1306 have uniform cross-sectional shapes along their length. In certain embodiments, the struts 1306 have non-uniform cross-sectional shapes along their length. In some embodiments, the proximal end portions 1310 of the struts 1306 have relatively large cross-sectional areas to facilitate pushability, and the intermediate portions 1312 of the struts 1306 have relatively small cross-sectional areas to facilitate flexibility.

In some embodiments, the apposition element 1302 has a first height from the catheter shaft at the first apposition state and a second height from the catheter shaft at the second apposition state, and the second height is greater than the first height. In certain embodiments, the first height is a negative value (that is, the apposition element 1302 is retracted relative to the outer surface of the catheter shaft 1304). In some embodiments, the first height is in a range of −2 mm to 1 mm. In certain embodiments, the second height is in a range of 5 mm to 25 mm. In certain embodiments, each strut 1306 of the apposition element 1302 has the same first height from the catheter shaft 1304 at the first apposition state and the same second height from the catheter shaft 1304 at the second apposition state. In some embodiments, one or more struts 1306 of the apposition element 1302 has a different first height from the catheter shaft 1304 at the first apposition state compared to one or more other struts 1306 of the apposition element 1302, and/or one or more struts 1306 of the apposition element 1302 has a different second height from the catheter shaft 1304 at the second apposition state compared to one or more other struts 1306 of the apposition element 1302. In some embodiments, the second height of the apposition element 1302 may be user-selectable. For example, in certain embodiments a user may move the proximal end portions 1310 of the struts 1306 by certain amounts relative to the catheter shaft 1304 to cause the apposition element 1302 to occupy certain second heights. In some embodiments, the shunting catheter 1300 may include an indicator (not shown) that associates certain amounts of movement of the proximal end portions 1310 of the struts 1306 with certain second heights of the apposition element 1302. In certain embodiments, moving the struts 1306 to the second apposition state and/or into contact with a vessel wall may provide haptic feedback to a user.

In some embodiments and as illustrated, the struts 1306 are disposed substantially non-symmetrically relative to the catheter shaft 1304. In other embodiments, the struts 1306 are disposed substantially symmetrically relative to the catheter shaft 1304. In certain embodiments, the struts 1306 are made of one or more materials that facilitate extension away from the catheter shaft 1304, such as a shape memory material set in the second apposition state. In some embodiments, the struts 1306 are made of nitinol, or one or more other materials, such as stainless steel, titanium, platinum-iridium, or cobalt-chromium.

In some embodiments and as shown in FIGS. 13D and 13E, the catheter shaft 1304 further includes a main lumen 1316 in which the shunting element is translatably disposed and a guidewire lumen 1318 in which a guidewire (not shown) is translatably received.

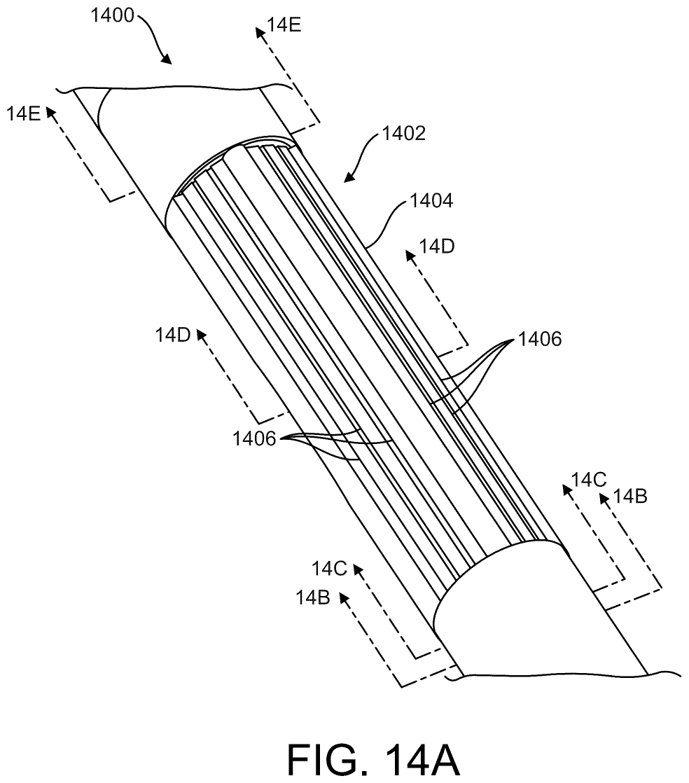

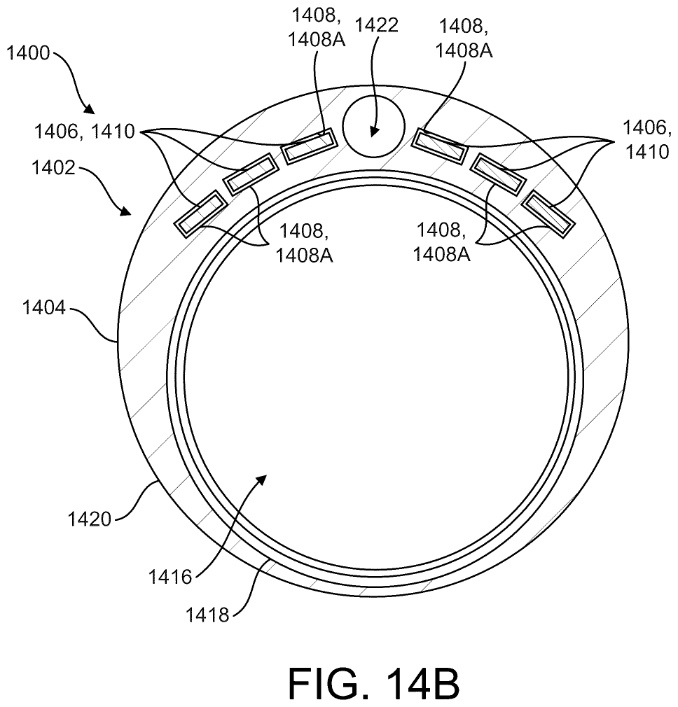

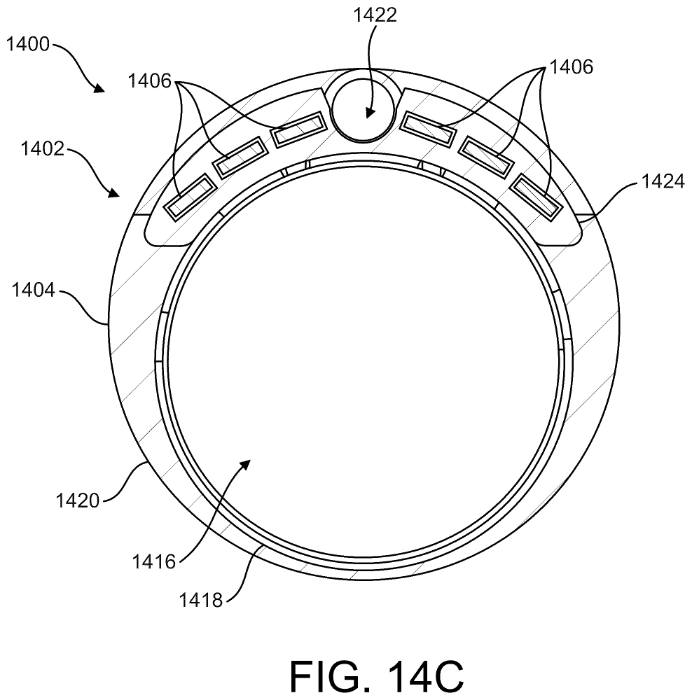

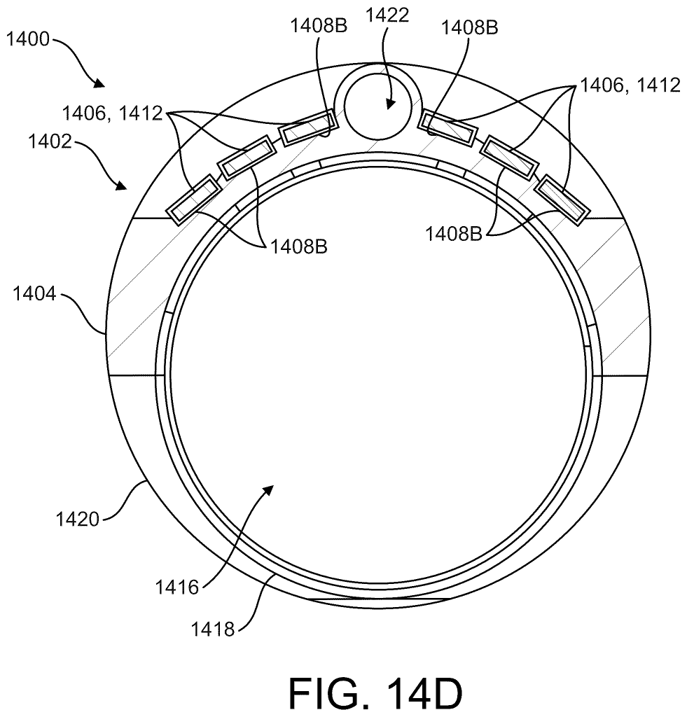

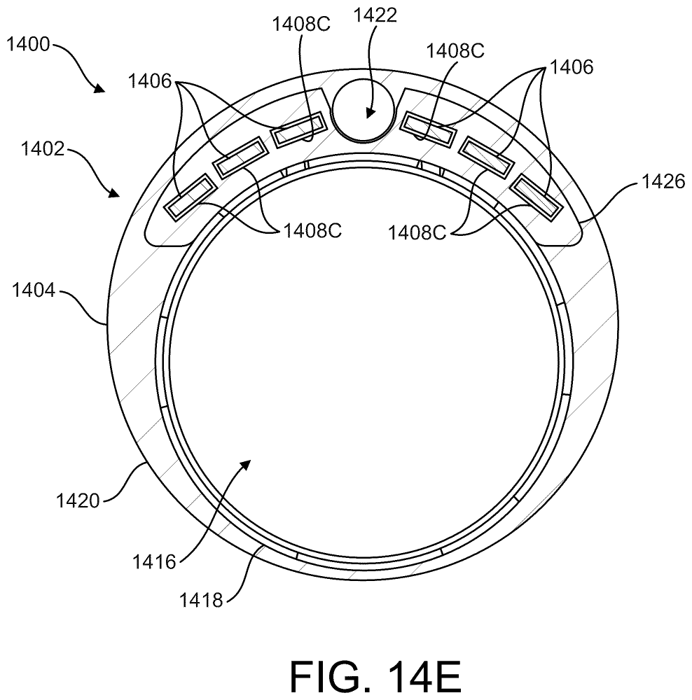

FIGS. 14A-14E are schematic diagrams of views of some examples of a shunting catheter 1400, in accordance with embodiments of the present disclosure. FIG. 14A illustrates an apposition element 1402 of the shunting catheter 1400 in a first apposition state, and FIGS. 14B-14E illustrate cross-sectional views of the apposition element 1402 and a catheter shaft 1404 of the shunting catheter 1400 in the first apposition state. According to certain embodiments, the apposition element 1402 is retracted against the catheter shaft 1404 at the first apposition state. In some embodiments, the apposition element 1402 extends outwardly from the catheter shaft 1404 at a second apposition state (not shown). In certain embodiments, the apposition element 1402 can appose to a wall of the vessel (for example, the front wall or back wall of the CS, a left atrium wall, a right atrium wall, etc.) at the second apposition state, for example, to help position and/or stabilize a shunting element (not shown) of the shunting catheter 1400.

According to some embodiments, the apposition element 1402 includes a plurality of movable struts 1406, which may be a plurality of wires. In some embodiments, the struts 1406 together define an expandable cage. In some embodiments, the spaces between adjacent struts 1406 allow blood flow through the apposition element 1402 in the second apposition state, thus reducing the risk of thrombus formation caused by any occlusion in the vessel.

In certain embodiments and as shown specifically in FIG. 14B, the catheter shaft 1404 includes one or more apposition element lumens 1408, and a lumen 1408 includes and/or carries one of the struts 1406 of the apposition element 1402. In some embodiments, a lumen 1408 includes a first lumen section 1408A (FIG. 14B), a second lumen section 1408B (FIG. 14D), and a third lumen section 1408C (FIG. 14E). In certain embodiments, the second lumen section 1408B is between the first lumen section 1408A and the third lumen section 1408C. In some embodiments, the third lumen section 1408C is disposed at or proximate to a distal end of the shunting catheter 1400. In certain embodiments, the first lumen section 1408A and/or the third lumen section 1408C are enclosed structure. In certain embodiments, a cross sectional shape of the first lumen section 1408A (e.g., as illustrated in FIG. 14B) and/or the third lumen section 1408C (e.g., as illustrated in FIG. 14E) is a close shape. In some embodiments, the second lumen section 1408B (e.g., as illustrated in FIG. 14D) is an open structure. In certain embodiments, a cross sectional shape of the second lumen section 1408B is not a closed shape.

In some embodiments, a proximal end portion 1410 (FIG. 14B) of each strut 1406 is movable in one of the lumens 1408, and a distal end portion (not shown) of each strut 1406 is inhibited from moving distally relative to the catheter shaft 1404. In certain embodiments, a distal end portion is secured to the catheter shaft 1404 within a distal lumen section 1408C, for example, via an adhesive. In some embodiments, a distal end portion is not secured to the catheter shaft 1404, although the distal end portions contact the catheter shaft 1404 within the distal lumens such that distal movement of the distal end portions of the struts 1406 relative to the catheter shaft 1404 is inhibited.

In certain embodiments and as illustrated, intermediate portions 1412 (FIG. 14D) of the struts 1406 are exposed to the exterior of the shunting catheter 1400 between the apposition element lumens 1408 and the distal lumens. In certain embodiments, moving the proximal end portions 1410 of the struts 1406 distally relative to the catheter shaft 1404 causes the intermediate portions 1412 of the struts 1406 to bow and extend outwardly from the catheter shaft 1404 at the second apposition state.

In certain embodiments and as illustrated, the apposition element 1402 includes six struts 1406. In other embodiments, the apposition element 1402 includes a different number of struts 1406, such as one, two, three, four, five, seven, eight, nine, ten or more struts 1406. In some embodiments, the struts 1406 have non-circular cross-sectional shapes, for example and as illustrated, rectangular cross-sectional shapes. In some embodiments, struts 1406 with non-circular cross-sectional shapes have preferential bowing axes (for example, parallel to the long sides of a rectangular cross-sectional shape) to guide movement of the struts 1406 from the first apposition state to the second apposition state. In some embodiments, the struts 1406 have circular cross-sectional shapes. In some embodiments, the struts 1406 have uniform cross-sectional shapes along their length. In certain embodiments, the struts 1406 have non-uniform cross-sectional shapes along their length. In some embodiments, the proximal end portions 1410 of the struts 1406 have relatively large cross-sectional areas to facilitate pushability, and the intermediate portions 1412 of the struts 1406 have relatively small cross-sectional areas to facilitate flexibility.

In some embodiments, the apposition element 1402 has a first height from the catheter shaft at the first apposition state and a second height from the catheter shaft at the second apposition state, and the second height is greater than the first height. In certain embodiments, the first height is a negative value (that is, the apposition element 1402 is retracted relative to the outer surface of the catheter shaft 1404). In some embodiments, the first height is in a range of −2 mm to 1 mm. In certain embodiments, the second height is in a range of 5 mm to 25 mm. In certain embodiments, each strut 1406 of the apposition element 1402 has the same first height from the catheter shaft 1404 at the first apposition state and the same second height from the catheter shaft 1404 at the second apposition state. In some embodiments, one or more struts 1406 of the apposition element 1402 has a different first height from the catheter shaft 1404 at the first apposition state compared to one or more other struts 1406 of the apposition element 1402, and/or one or more struts 1406 of the apposition element 1402 has a different second height from the catheter shaft 1404 at the second apposition state compared to one or more other struts 1406 of the apposition element 1402. In some embodiments, the second height of the apposition element 1402 may be user-selectable. For example, in certain embodiments a user may move the proximal end portions 1410 of the struts 1406 by certain amounts relative to the catheter shaft 1404 to cause the apposition element 1402 to occupy certain second heights. In some embodiments, the shunting catheter 1400 may include an indicator (not shown) that associates certain amounts of movement of the proximal end portions 1410 of the struts 1406 with certain second heights of the apposition element 1402. In certain embodiments, moving the struts 1406 to the second apposition state and/or into contact with a vessel wall may provide haptic feedback to a user.

In some embodiments and as illustrated, the struts 1406 are disposed substantially non-symmetrically relative to the catheter shaft 1404. In other embodiments, the struts 1406 are disposed substantially symmetrically relative to the catheter shaft 1404. In certain embodiments, the struts 1406 are made of one or more materials that facilitate extension away from the catheter shaft 1404, such as a shape memory material set in the second apposition state. In some embodiments, the struts 1406 are made of nitinol, or one or more other materials, such as stainless steel, titanium, platinum-iridium, or cobalt-chromium.

In some embodiments and as shown in FIGS. 14C and 14E, the catheter shaft 1404 further includes a main lumen 1416 in which the shunting element is translatably disposed. In certain embodiments, the main lumen 1416 is formed by an inner tube 1418, for example a stainless steel hypotube. In some embodiments, an outer layer 1420 surrounds the inner tube 1418 and forms the apposition element lumens 1408. In certain embodiments and as illustrated, the outer layer 1420 forms a guidewire lumen 1422 in which a guidewire (not shown) is translatably received. In some embodiments, the outer layer 1420 is constructed of a reflow polymer material. In some embodiments and as illustrated, a proximal apposition element alignment guide 1424 (e.g., FIG. 14C) and a distal apposition element alignment guide 1426 (e.g., FIG. 14E) are coupled to the inner tube 1418 and embedded in the outer layer 1420. In some embodiments, the struts 1406 slide through the proximal apposition element alignment guide 1424 and/or the distal apposition element alignment guide 1426 when transitioning from the first apposition state to the second apposition state. In certain embodiments, the apposition element alignment guides 1424, 1426 are constructed of a stiffer material than the outer layer 1420 (for example, stainless steel) to facilitate proper positioning of the struts 1406 in the second apposition state. In certain embodiments, the apposition element alignment guides 1424, 1426 are constructed of a radiopaque material or include a radiopaque coating to facilitate positioning the catheter 1400 under fluoroscopy.

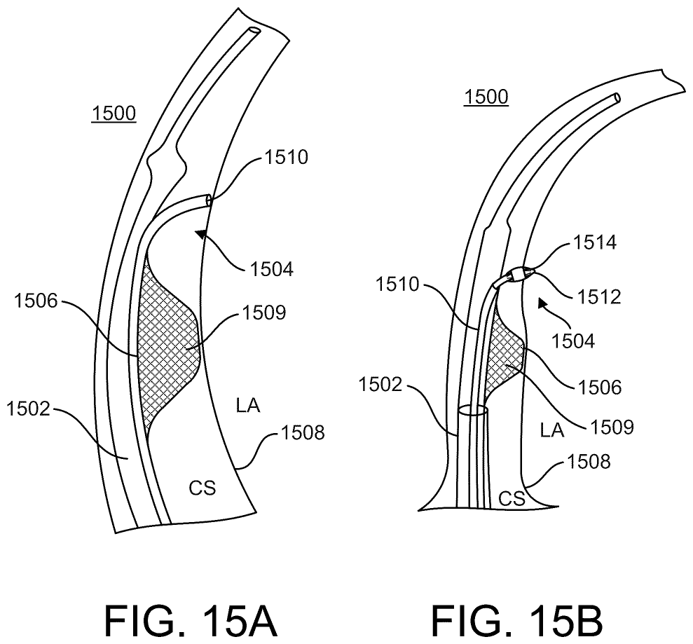

FIGS. 15A-15B are schematic diagrams of side views of an example of a shunting catheter 1500, according to certain embodiments of the present disclosure. FIGS. 15A-15B are merely examples. One of the ordinary skilled in the art would recognize many variations, alternatives, and modifications. In some embodiments and as shown, the shunting catheter 1500 includes a catheter shaft 1502 and a shunting element 1504, more specifically an ablation assembly 1504.

In some embodiments, the ablation assembly 1504 is disposed within the catheter shaft 1502 at a first state (for example, during deployment, during deployment to position the ablation assembly 1504). In some embodiments, the ablation assembly 1504 is extended from the catheter shaft 1502 at a second state (for example, during shunting).

According to certain embodiments, the shunting catheter 1500 includes an apposition element 1506 configured to be disposed within the catheter shaft 1502 at a first state (for example, during deployment), and protrudes from the catheter shaft 1502 at a second state (for example, during shunting). According to some embodiments, for example during the tracking of the shunting catheter 1500 to a target location in a patient's CS, the ablation assembly 1504 may be translated out of the catheter shaft 1502 to puncture a target location on a wall 1508 (for example, a vessel wall between a patient's CS and LA). In embodiments, the apposition element 1506 is made of a flexible material and configured to appose the wall 1508 during shunting. In some embodiments and as illustrated, the apposition element 1506 extends substantially non-symmetrically from the catheter shaft 1502. In other embodiments, the apposition element 1506 extends substantially symmetrically from the catheter shaft 1502. In some embodiments, the apposition element 1506 provides stability to the shunting catheter 1500 during deployment and/or shunting. In some embodiments, the apposition element 1506 includes a plurality of movable struts 1509, more specifically a plurality of movable wires 1509. In certain embodiments, the spaces between adjacent wires 1509 allow blood flow through the apposition element 1506, thus reducing the risk of thrombus formation caused by any occlusion in the vessel.

In some embodiments, the ablation assembly 1504 includes a crimping shaft 1510 having a predetermined curve, a puncture element 1512, and a shunting element 1514. In some embodiments, the ablation assembly 1504 may have a telescoping feature (for example, the puncture element 1512 and the shunting element 1514 are retractable into the crimping shaft 1510) to allow for the blunt end of the crimping shaft 1510 to contact the wall 1508 before the puncture element 1512 is translated forward to make contact with the wall 1508. In certain embodiments, the telescoping feature of the ablation assembly 1504 allows for a safe delivery of the puncture element 1512 to the target location.

In some embodiments, for example as shown in FIG. 15A, the ablation assembly 1504 has a first deployed state where the ablation assembly 1504 is extended from the catheter shaft 1502, and the puncture element 1512 and the shunting element 1514 are retracted and crimped inside the crimping shaft 1510. In certain embodiments and as shown, the distal end of the ablation assembly 1504 is blunt during the first deployment state, such that if adjustment of position is needed, the wall surrounding the target location would only make contact with a blunt surface.

In some embodiments, for example as shown in FIG. 15B, the ablation assembly 1504 has a second deployed state where the ablation assembly 1504 is extended from the catheter shaft 1502, and the puncture element 1512 and the shunting element 1514 are all protruded from the crimping shaft 1510. In certain embodiments, the puncture element 1512 punctures an opening in the wall 1508 in the patient upon moving from the first deployed state to the second deployed state. In certain embodiments, the ablation assembly 1504 is expanded in the second deployed state and thereby enlarges the opening in the wall 1508 in the patient. In certain embodiments, the ablation assembly 1504 ablates tissue at the opening in the wall 1508 in the patient by delivering ablation energy (such as radiofrequency (RF) energy, phased RF energy, cryogenic energy, thermal energy, pulse energy, laser energy, microwave energy, ultrasound energy, and/or the like) to the tissue.

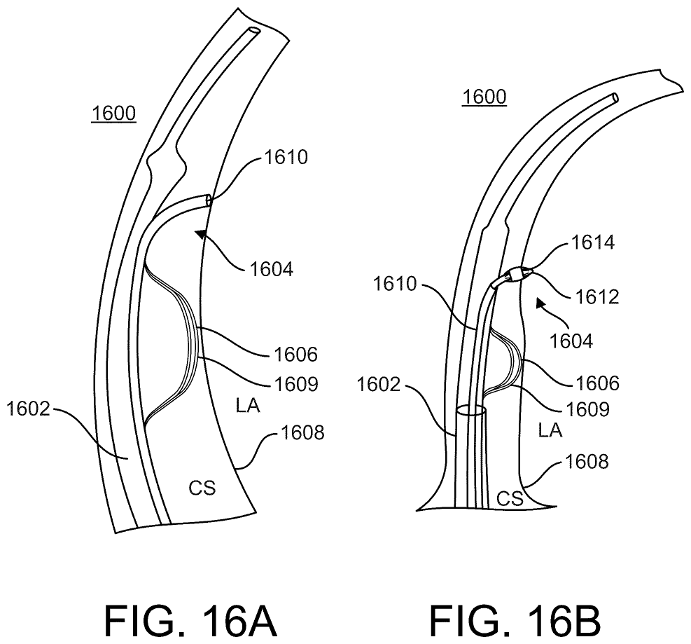

FIGS. 16A-16B are schematic diagrams of side views of another example of a shunting catheter 1600, according to certain embodiments of the present disclosure. FIGS. 16A-16B are merely examples. One of the ordinary skilled in the art would recognize many variations, alternatives, and modifications. In some embodiments and as shown, the shunting catheter 1600 includes a catheter shaft 1602 and a shunting element 1604, more specifically an ablation assembly 1604.

In some embodiments, the ablation assembly 1604 is disposed within the catheter shaft 1602 at a first state (for example, during deployment, during deployment to position the ablation assembly 1604). In some embodiments, the ablation assembly 1604 is extended from the catheter shaft 1602 at a second state (for example, during shunting).

According to certain embodiments, the shunting catheter 1600 includes an apposition element 1606 configured to be disposed within the catheter shaft 1602 at a first state (for example, during deployment), and protrudes from the catheter shaft 1602 at a second state (for example, during shunting). According to some embodiments, for example during the tracking of the shunting catheter 1600 to a target location in a patient's CS, the ablation assembly 1604 may be translated out of the catheter shaft 1602 to puncture a target location on a wall 1608 (for example, a vessel wall between a patient's CS and LA). In embodiments, the apposition element 1606 is made of a flexible material and configured to appose the wall 1608 during shunting. In some embodiments, the apposition element 1606 extends substantially symmetrically from the catheter shaft 1602. In other embodiments and as illustrated, the apposition element 1606 extends substantially non-symmetrically from the catheter shaft 1602. In some embodiments, the apposition element 1606 provides stability to the shunting catheter 1600 during deployment and/or shunting. In some embodiments, the apposition element 1606 includes a plurality of movable struts 1609. In certain embodiments, the spaces between adjacent struts 1609 allow blood flow through the apposition element 1606, thus reducing the risk of thrombus formation caused by any occlusion in the vessel.

In some embodiments, the ablation assembly 1604 includes a crimping shaft 1610 having a predetermined curve, a puncture element 1612, and a shunting element 1614. In some embodiments, the ablation assembly 1604 may have a telescoping feature (for example, the puncture element 1612 and the shunting element 1614 are retractable into the crimping shaft 1610) to allow for the blunt end of the crimping shaft 1610 to contact the wall 1608 before the puncture element 1612 is translated forward to make contact with the wall 1608. In certain embodiments, the telescoping feature of the ablation assembly 1604 allows for a safe delivery of the puncture element 1612 to the target location.

In some embodiments, for example as shown in FIG. 16A, the ablation assembly 1604 has a first deployed state where the ablation assembly 1604 is extended from the catheter shaft 1602, and the puncture element 1612 and the shunting element 1614 are retracted and crimped inside the crimping shaft 1610. In certain embodiments and as shown, the distal end of the ablation assembly 1604 is blunt during the first deployment state, such that if adjustment of position is needed, the wall surrounding the target location would only make contact with a blunt surface.

In some embodiments, for example as shown in FIG. 16B, the ablation assembly 1604 has a second deployed state where the ablation assembly 1604 is extended from the catheter shaft 1602, and the puncture element 1612 and the shunting element 1614 are all protruded from the crimping shaft 1610. In certain embodiments, the puncture element 1612 punctures an opening in the wall 1608 in the patient upon moving from the first deployed state to the second deployed state. In certain embodiments, the ablation assembly 1604 is expanded in the second deployed state and thereby enlarges the opening in the wall 1608 in the patient. In certain embodiments, the ablation assembly 1604 ablates tissue at the opening in the wall 1608 in the patient by delivering ablation energy (such as radiofrequency (RF) energy, phased RF energy, cryogenic energy, thermal energy, pulse energy, laser energy, microwave energy, ultrasound energy, and/or the like) to the tissue.

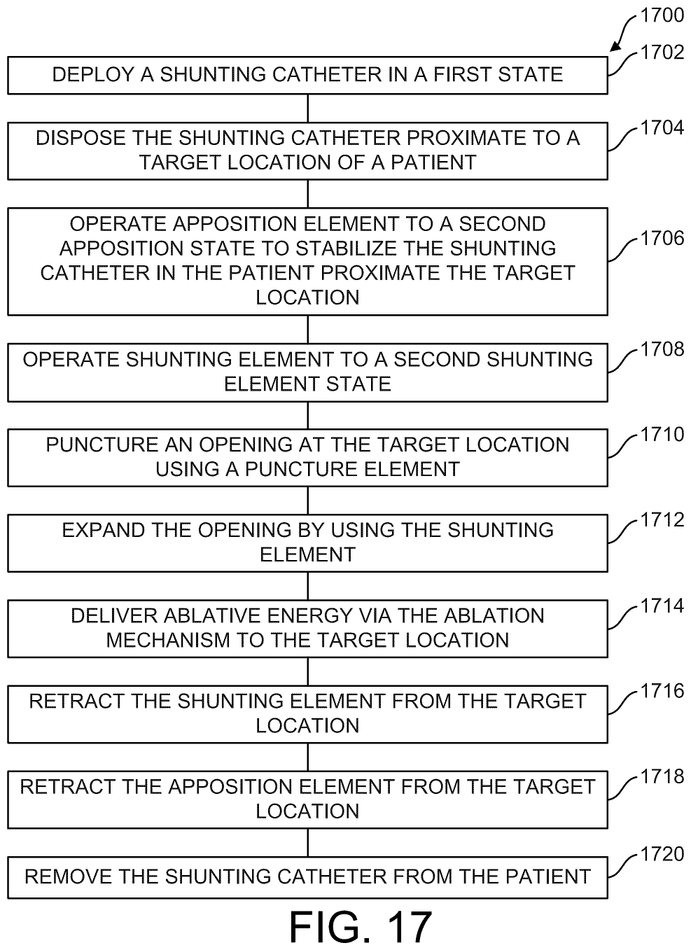

FIG. 17 is a flow diagram illustrating an example method 1700 of creating a shunt in a patient, in accordance with embodiments of the present disclosure. Aspects of embodiments of the method may be performed, for example, by a shunting catheter system or a controller (for example, the system 104 in FIG. 1, the controller 112 in FIG. 1). One or more steps of method are optional and/or can be modified by one or more steps of other embodiments described herein. Additionally, one or more steps of other embodiments described herein may be added to the method. In some embodiments, the shunt may be formed in a CS of a patient. In certain embodiments, the shunt includes an opening between a patient's CS and LA.

At step 1702, the method 1700 includes deploying a shunting catheter in a first state. In certain embodiments, deploying the shunting catheter in the first state includes deploying the shunting catheter in a first shunting element state and a first apposition state. In some embodiments, a shunting shaft of the shunting catheter is disposed in a shaft lumen of a catheter shaft at the first shunting element state. In certain embodiments, an apposition element of the shunting catheter is disposed at the catheter shaft at the first apposition state. According to certain embodiments, the apposition element includes a plurality of movable struts. In some embodiments, the apposition element is disposed within an outer shaft disposed outside of the catheter shaft at the first apposition state. In certain embodiments, deploying the shunting catheter includes inserting the shunting catheter through a superior vena cava of a patient into a CS of the patient. In certain embodiments, deploying the shunting catheter includes inserting the shunting catheter through an inferior vena cava of a patient into a CS of the patient.

At step 1704, the method 1700 includes disposing the shunting catheter proximate to a target location of a patient. At step 1706, the method 1700 includes operating the apposition element to a second apposition state in which the apposition element extends from the catheter shaft to contact tissue of the patient and thereby stabilize the shunting catheter proximate to the target location of the patient. In certain embodiments, the apposition element is disposed substantially symmetrically around the catheter shaft, the target location is at a blood vessel of the patient, and operating the apposition element to the second apposition state causes the shunting catheter to be positioned substantially concentrically in the blood vessel. In other embodiments, the apposition element is disposed substantially non-symmetrically around the catheter shaft, the target location is at a blood vessel of the patient, and operating the apposition element to the second apposition state causes the shunting catheter to be positioned substantially non-concentrically in the blood vessel. According to some embodiments, operating the apposition element to the second apposition state includes permitting the apposition element to self-expand. In some embodiments, permitting the apposition element to self-expand includes retracting the outer shaft from the apposition element. In certain embodiments, permitting the apposition element to self-expand includes permitting the struts to self-move. In certain embodiments, operating the apposition element to the second apposition state includes permitting blood flow through the apposition element, more specifically between the struts.

At step 1708, the method 1700 includes operating the shunting element to a second shunting element state where the shunting element extends from the catheter shaft. In certain embodiments, the shunting element is disposed in the shunting shaft. In some embodiments, the shunting shaft is extended from a side opening of the catheter shaft. In certain embodiments, the shunting shaft is extended from an end of the catheter shaft. In some embodiments, operating the shunting element to the second shunting element state further includes retracting a crimping shaft from a puncture element of the shunting element.

At step 1710, the method 1700 includes puncturing, using the puncture element, an opening at the target location. In some embodiments, the target location is at a CS of a patient. In some embodiments, the puncture element physically contacts tissue to puncture an opening at the target location in the patient. In certain embodiments, the puncture element includes a hollow needle, and puncturing the opening at the target location includes using the hollow needle. In some embodiments, the puncture element includes a plurality of voids that facilitate bending of the puncture element in the second shunting element state. In certain embodiments, the puncture element receives energy (for example, electrical energy) and delivers ablation energy (such as radiofrequency (RF) energy, phased RF energy, cryogenic energy, thermal energy, pulse energy, laser energy, ultrasound energy, microwave energy, and/or the like) to tissue to puncture an opening at the target location in the patient.

At step 1712, the method 1700 includes expanding the opening using the shunting element. In some embodiments, expanding the opening includes permitting movable struts of the shunting element to self-move in the second shunting element state. In some embodiments, expanding the opening includes moving the movable struts by operating an actuator, for example inflating a balloon carried within the shunting element.

At step 1714, the method 1700 includes delivering ablation energy (such as radiofrequency (RF) energy, phased RF energy, cryogenic energy, thermal energy, pulse energy, laser energy, ultrasound energy, microwave energy, and/or the like) via the shunting element to tissue at the target location. In some embodiments, the shunting element delivers the ablation energy while expanded in the second shunting element state. In some embodiments, delivering the ablation energy to the tissue at the target location solidifies the opening at the target location.

At step 1716, the method 1700 includes retracting the puncture element and the shunting element from the tissue at the target location in the patient. In some embodiments, retracting the puncture element and the shunting element from the tissue at the target location includes operating the shunting element to the first shunting element state. In certain embodiments, retracting the puncture element and the shunting element includes moving the puncture element and the shunting element into the crimping shaft. In certain embodiments, retracting the puncture element and the shunting element includes compressing the shunting element in the crimping shaft.

At step 1718, the method 1700 includes retracting the apposition element from a location proximate to the target location in the patient. In certain embodiments, retracting the apposition element includes operating the apposition element to the first apposition state. In some embodiments, retracting the apposition element includes covering the apposition element with the outer shaft.

At step 1720, the method 1700 includes removing the shunting catheter from the patient. In some embodiments, the method 1700 may include removing the shunting catheter, which includes removing the catheter shaft, the shunting element, and the apposition element. In certain embodiments, the method 1700 does not leave any implant device at the target location. In some embodiments, the formed shunt is an opening between a CS and a LA of a patient. In certain embodiments, the shunting catheter is removed from the CS of the patient. In certain embodiments, the formed shunt is an opening that does not include an implant (for example, a frame or structure to support an opening). In some embodiments, the shunt includes an opening between the CS and the LA of a patient, where the shunt does not include an implant.

According to some embodiments, the method 1700 includes generating a shunt using a puncture element and a shunting element of a shunting catheter. In certain embodiments, the shunt includes an expanded opening between the CS and LA of a patient. In some embodiments, the shunt does not include any implant.

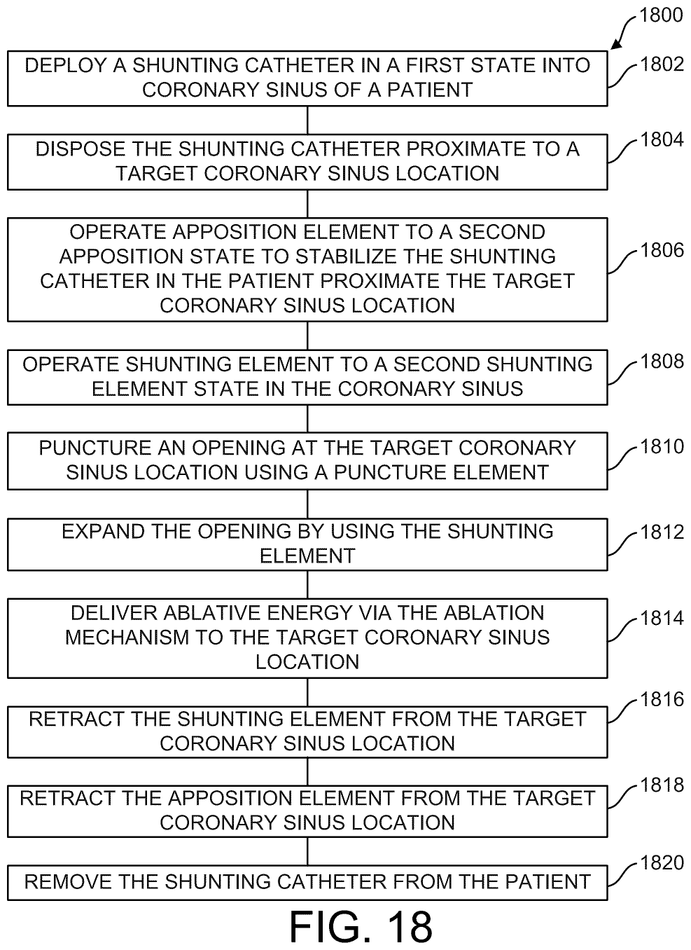

FIG. 18 is a flow diagram illustrating an example method 1800 of creating a shunt in a patient, in accordance with embodiments of the present disclosure. Aspects of embodiments of the method may be performed, for example, by a shunting catheter system or a controller (for example, the system 104 in FIG. 1, the controller 112 in FIG. 1). One or more steps of method are optional and/or can be modified by one or more steps of other embodiments described herein. Additionally, one or more steps of other embodiments described herein may be added to the method. In some embodiments, the shunt may be formed in a CS of a patient. In certain embodiments, the shunt includes an opening between a patient's CS and LA.

At step 1802, the method 1800 includes deploying a shunting catheter in a first state into a CS of a patient. In certain embodiments, deploying the shunting catheter includes inserting the shunting catheter through a superior vena cava of a patient into the CS of the patient. In certain embodiments, deploying the shunting catheter includes inserting the shunting catheter through an inferior vena cava of a patient into the CS of the patient. In certain embodiments, deploying the shunting catheter in the first state includes deploying the shunting catheter in a first shunting element state and a first apposition state. In some embodiments, a shunting shaft of the shunting catheter is disposed in a shaft lumen of a catheter shaft at the first shunting element state. In certain embodiments, an apposition element of the shunting catheter is disposed at the catheter shaft at the first apposition state. According to certain embodiments, the apposition element includes a plurality of movable struts. In some embodiments, the apposition element is disposed within an outer shaft disposed outside of the catheter shaft at the first apposition state.

At step 1804, the method 1800 includes disposing the shunting catheter proximate to a target CS location of the patient. At step 1806, the method 1800 includes operating the apposition element to a second apposition state in which the apposition element extends from the catheter shaft to contact tissue of the patient and thereby stabilize the shunting catheter proximate to the target CS location of the patient. In certain embodiments, the apposition element is disposed substantially symmetrically around the catheter shaft, and operating the apposition element to the second apposition state causes the shunting catheter to be positioned substantially concentrically in the CS. In other embodiments, the apposition element is disposed substantially non-symmetrically around the catheter shaft, and operating the apposition element to the second apposition state causes the shunting catheter to be positioned substantially non-concentrically in the CS. According to some embodiments, operating the apposition element to the second apposition state includes permitting the apposition element to self-expand. In some embodiments, permitting the apposition element to self-expand includes retracting the outer shaft from the apposition element. In certain embodiments, permitting the apposition element to self-expand includes permitting the struts to self-move. In certain embodiments, operating the apposition element to the second apposition state includes permitting blood flow through the apposition element, more specifically between the struts.

At step 1808, the method 1800 includes operating the shunting element to a second shunting element state where the shunting element extends from the catheter shaft. In certain embodiments, the shunting element is disposed in the shunting shaft. In some embodiments, the shunting shaft is extended from a side opening of the catheter shaft. In certain embodiments, the shunting shaft is extended from an end of the catheter shaft. In some embodiments, operating the shunting element to the second shunting element state further includes retracting a crimping shaft from a puncture element of the shunting element.

At step 1810, the method 1800 includes puncturing, using the puncture element, an opening at the target CS location. In some embodiments, the puncture element physically contacts tissue to puncture an opening at the target CS location. In certain embodiments, the puncture element includes a hollow needle, and puncturing the opening at the target CS location includes using the hollow needle. In some embodiments, the puncture element includes a plurality of voids that facilitate bending of the puncture element in the second shunting element state. In certain embodiments, the puncture element receives energy (for example, electrical energy) and delivers ablation energy (such as radiofrequency (RF) energy, phased RF energy, cryogenic energy, thermal energy, pulse energy, laser energy, ultrasound energy, microwave energy, and/or the like) to tissue to puncture an opening at the target CS location.

At step 1812, the method 1800 includes expanding the opening using the shunting element. In some embodiments, expanding the opening includes permitting movable struts of the shunting element to self-move in the second shunting element state. In some embodiments, expanding the opening includes moving the movable struts by operating an actuator, for example inflating a balloon carried within the shunting element.

At step 1814, the method 1800 includes delivering ablation energy (such as radiofrequency (RF) energy, phased RF energy, cryogenic energy, thermal energy, pulse energy, laser energy, ultrasound energy, microwave energy, and/or the like) via the shunting element to tissue at the target CS location. In some embodiments, the shunting element delivers the ablation energy while expanded in the second shunting element state. In some embodiments, delivering the ablation energy to the tissue at the target CS location solidifies the opening at the target CS location.

At step 1816, the method 1800 includes retracting the puncture element and the shunting element from the tissue at the target CS location in the patient. In some embodiments, retracting the puncture element and the shunting element from the tissue at the target CS location includes operating the shunting element to the first shunting element state. In certain embodiments, retracting the puncture element and the shunting element includes moving the puncture element and the shunting element into the crimping shaft. In certain embodiments, retracting the puncture element and the shunting element includes compressing the shunting element in the crimping shaft.

At step 1818, the method 1800 includes retracting the apposition element from tissue at the target CS location in the patient. In certain embodiments, retracting the apposition element includes operating the apposition element to the first apposition state. In some embodiments, retracting the apposition element includes covering the apposition element with the outer shaft.

At step 1820, the method 1800 includes removing the shunting catheter from the patient. In some embodiments, the method 1800 may include removing the shunting catheter, which includes removing the catheter shaft, the shunting element, and the apposition element. In certain embodiments, the method 1800 does not leave any implant device at the target CS location. In some embodiments, the formed shunt is an opening between a CS and a LA of a patient. In certain embodiments, the formed shunt is an opening that does not include an implant (for example, a frame or structure to support an opening). In some embodiments, the shunt includes an opening between the CS and the LA of a patient, where the shunt does not include an implant.

According to some embodiments, the method 1800 includes generating a shunt using a puncture element and a shunting element of a shunting catheter. In certain embodiments, the shunt includes an expanded opening between the CS and LA of a patient. In some embodiments, the shunt does not include any implant.

In certain embodiments, a shunting catheter includes: a catheter shaft including a shaft lumen; a shunting element disposed in the shaft lumen at a first shunting element state and extended from the catheter shaft at a second shunting element state; and an apposition element disposed proximate to the shunting element, the apposition element disposed at the catheter shaft at a first apposition state and extending from the catheter shaft at a second apposition state.

In some embodiments, an outer shaft is disposed outside of at least a portion of the catheter shaft, and the apposition element is disposed within the outer shaft at the first apposition state.

In certain embodiments, the apposition element has a first height from the catheter shaft at the first apposition state and a second height from the catheter shaft at the second apposition state, and the second height is greater than the first height.

In some embodiments, the second height is at least two times the first height.

In certain embodiments, the second height is user-selectable.

In some embodiments, the apposition element is disposed substantially symmetrically around the catheter shaft.

In certain embodiments, the apposition element is disposed substantially non-symmetrically around the catheter shaft.

In some embodiments, the apposition element includes a plurality of braided wires.

In certain embodiments, the apposition element includes a plurality of movable struts.

In some embodiments, at least one strut of the plurality of movable struts includes at least one hole.

In certain embodiments, each strut of the plurality of movable struts includes a first strut portion and a second strut portion, the first strut portion and an axis defined by the catheter shaft form an expansion angle in the second apposition state, and the expansion angle is greater than zero degrees.

In some embodiments, the expansion angle is greater than 15 degrees.

In certain embodiments, the first strut portion and the second strut portion form a strut angle greater than zero degrees in the second apposition state.

In some embodiments, the catheter shaft further includes a plurality of apposition element lumens, and at least one strut of the plurality of movable struts is movably disposed in one of the apposition element lumens of the plurality of apposition element lumens.

In certain embodiments, at least one strut of the plurality of movable struts includes a proximal end portion, a distal end portion, and an intermediate portion disposed between the proximal end portion and the distal end portion, the intermediate portion generally aligns with the catheter shaft at the first apposition state, and the intermediate portion extends outwardly from the catheter shaft at the second apposition state.

In some embodiments, the apposition element is self-expandable.

In certain embodiments, the apposition element includes a first end and a second end, the first end being secured to the catheter shaft and the second end being disposed apart from the catheter shaft.

In some embodiments, the apposition element includes a first end and a second end, and the first end is movable relative to the catheter shaft and the second end being secured to the catheter shaft.

In certain embodiments, the apposition element includes at least one material selected from a group consisting of nitinol, stainless steel, titanium, platinum-iridium, and cobalt-chromium.

In some embodiments, the catheter shaft defines a first axis; the shunting element defines a second axis at the second shunting element state; and the second axis and the first axis form an angle greater than zero degrees.

In certain embodiments, the shunting element includes a puncture element.

In some embodiments, the puncture element includes a hollow needle.

In certain embodiments, the puncture element includes a plurality of voids, and the plurality of voids facilitate bending of the shunting element in the second shunting element state.

In some embodiments, a method for creating a shunt includes: deploying a shunting catheter in a patient, the shunting catheter including: a catheter shaft including a shaft lumen; a shunting element disposed in the shaft lumen at a first shunting element state; and an apposition element disposed proximate to the shunting element, the apposition element disposed at the catheter shaft at a first apposition state; disposing the shunting catheter proximate to a target location of the patient; operating the apposition element to a second apposition state, wherein the apposition element extends from the catheter shaft at the second apposition state and stabilizes the shunting catheter in the patient proximate the target location; operating the shunting element to a second shunting element state, wherein the shunting element extends from the catheter shaft at the second shunting element state; puncturing, using the shunting element, an opening at the target location of the patient; and expanding, using the shunting element, the opening.

In certain embodiments, the apposition element is disposed substantially symmetrically around the catheter shaft, the target location is at a blood vessel of the patient, and operating the apposition element to the second apposition state causes the shunting catheter to be positioned substantially concentrically in the blood vessel.

In some embodiments, the apposition element is disposed substantially non-symmetrically around the catheter shaft, the target location is at a blood vessel of the patient, and operating the apposition element to the second apposition state causes the shunting catheter to be positioned substantially non-concentrically in the blood vessel.

In certain embodiments, operating the apposition element to the second apposition state includes permitting the apposition element to self-expand.

In some embodiments, the shunting catheter further includes an outer shaft disposed outside of at least a portion of the catheter shaft, the apposition element is disposed within the outer shaft at the first apposition state, and permitting the apposition element to self-expand includes retracting the outer shaft from the apposition element.

In certain embodiments, operating the apposition element to the second apposition state includes permitting blood flow through the apposition element.

In some embodiments, the shunting element includes a puncture element, and puncturing the opening at the target location of the patient includes using the puncture element.

In certain embodiments, the puncture element includes a hollow needle, and puncturing the opening at the target location of the patient includes using the hollow needle.

In some embodiments, the puncture element includes a plurality of voids, and the plurality of voids facilitate bending of the shunting element in the second shunting element state.

In certain embodiments, the target location is at a coronary sinus of the patient.

In some embodiments, a shunting catheter system includes: a shunting catheter, including: a catheter shaft including a shaft lumen; a shunting element disposed in the shaft lumen at a first shunting element state and extended from the catheter shaft at a second shunting element state, the shunting element including a puncture element; an apposition element disposed proximate to the shunting element, the apposition element disposed at the catheter shaft at a first apposition state and extending from the catheter shaft at a second apposition state; and an energy source connected to the shunting catheter; and a controller connected to the energy source and including a processor; the processor is configured to control the energy source to deliver ablation energy to a target location of a patient via the puncture element.

In certain embodiments, the shunting catheter further includes an outer shaft disposed outside of at least a portion of the catheter shaft, and the apposition element is disposed within the outer shaft at the first apposition state.

In some embodiments, the apposition element is disposed substantially non-symmetrically around the catheter shaft.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

What is claimed is:

1. A shunting catheter, comprising:
a catheter shaft including a shaft lumen;
a shunting element disposed in the shaft lumen at a first shunting element state and extended from the catheter shaft at a second shunting element state; and
an apposition element disposed proximate to the shunting element, the apposition element disposed at the catheter shaft at a first apposition state and extending from the catheter shaft at a second apposition state.

2. The shunting catheter of claim 1, further comprising an outer shaft disposed outside of at least a portion of the catheter shaft, wherein the apposition element is disposed within the outer shaft at the first apposition state.

3. The shunting catheter of claim 1, wherein the apposition element has a first height from the catheter shaft at the first apposition state and a second height from the catheter shaft at the second apposition state, wherein the second height is greater than the first height.

4. The shunting catheter of claim 3, wherein the second height is at least two times the first height.

5. The shunting catheter of claim 3, wherein the second height is user-selectable.

6. The shunting catheter of claim 1, wherein the apposition element is disposed substantially symmetrically around the catheter shaft.

7. The shunting catheter of claim 1, wherein the apposition element is disposed substantially non-symmetrically around the catheter shaft.

8. The shunting catheter of claim 1, wherein the apposition element comprises a plurality of braided wires.

9. The shunting catheter of claim 1, wherein the apposition element comprises a plurality of movable struts.

10. The shunting catheter of claim 9, wherein at least one strut of the plurality of movable struts includes at least one hole.

11. The shunting catheter of claim 9, wherein each strut of the plurality of movable struts includes a first strut portion and a second strut portion, wherein the first strut portion and an axis defined by the catheter shaft form an expansion angle in the second apposition state, and the expansion angle is greater than zero degrees.

12. The shunting catheter of claim 9, wherein the catheter shaft further includes a plurality of apposition element lumens, and at least one strut of the plurality of movable struts is movably disposed in one of the apposition element lumens of the plurality of apposition element lumens.

13. The shunting catheter of claim 1, wherein the apposition element is self-expandable.

14. The shunting catheter of claim 1, wherein the apposition element includes a first end and a second end, the first end being secured to the catheter shaft and the second end being disposed apart from the catheter shaft.

15. The shunting catheter of claim 1, wherein the apposition element includes a first end and a second end, the first end being movable relative to the catheter shaft and the second end being secured to the catheter shaft.

16. The shunting catheter of claim 1, wherein the catheter shaft defines a first axis; wherein the shunting element defines a second axis at the second shunting element state; wherein the second axis and the first axis form an angle greater than zero degrees.

17. The shunting catheter of claim 1, wherein the shunting element includes a puncture element.

18. The shunting catheter of claim 17, wherein the puncture element includes a hollow needle.

19. The shunting catheter of claim 17, wherein the puncture element includes a plurality of voids, the plurality of voids facilitating bending of the shunting element in the second shunting element state.

20. A method for creating a shunt, comprising:
deploying a shunting catheter in a patient, the shunting catheter comprising:
a catheter shaft comprising a shaft lumen;
a shunting element disposed in the shaft lumen at a first shunting element state; and
an apposition element disposed proximate to the shunting element, the apposition element disposed at the catheter shaft at a first apposition state;
disposing the shunting catheter proximate to a target location of the patient;
operating the apposition element to a second apposition state, wherein the apposition element extends from the catheter shaft at the second apposition state and stabilizes the shunting catheter in the patient proximate the target location;
operating the shunting element to a second shunting element state, wherein the shunting element extends from the catheter shaft at the second shunting element state;
puncturing, using the shunting element, an opening at the target location of the patient; and
expanding, using the shunting element, the opening.

21. The method of claim 20, wherein the apposition element is disposed substantially symmetrically around the catheter shaft, wherein the target location is at a blood vessel of the patient, and operating the apposition element to the second apposition state causes the shunting catheter to be positioned substantially concentrically in the blood vessel.

22. The method of claim 20, wherein the apposition element is disposed substantially non-symmetrically around the catheter shaft, wherein the target location is at a blood vessel of the patient, and operating the apposition element to the second apposition state causes the shunting catheter to be positioned substantially non-concentrically in the blood vessel.

23. The method of claim 20, wherein operating the apposition element to the second apposition state comprises permitting the apposition element to self-expand.

24. The method of claim 23, wherein the shunting catheter further comprises an outer shaft disposed outside of at least a portion of the catheter shaft, the apposition element is disposed within the outer shaft at the first apposition state, and permitting the apposition element to self-expand comprises retracting the outer shaft from the apposition element.

25. The method of claim 20, wherein operating the apposition element to the second apposition state comprises permitting blood flow through the apposition element.

26. The method of claim 20, wherein the shunting element comprises a puncture element, and puncturing the opening at the target location of the patient comprises using the puncture element.

27. The method of claim 20, wherein the target location is at a coronary sinus of the patient.

28. A shunting catheter system, comprising:
a shunting catheter, comprising:
a catheter shaft including a shaft lumen;
a shunting element disposed in the shaft lumen at a first shunting element state and extended from the catheter shaft at a second shunting element state, the shunting element including a puncture element;
an apposition element disposed proximate to the shunting element, the apposition element disposed at the catheter shaft at a first apposition state and extending from the catheter shaft at a second apposition state; and
an energy source connected to the shunting catheter; and
a controller connected to the energy source and comprising a processor;
wherein the processor is configured to control the energy source to deliver ablation energy to a target location of a patient via the puncture element.

29. The shunting catheter system of claim 28, wherein the shunting catheter further includes an outer shaft disposed outside of at least a portion of the catheter shaft, wherein the apposition element is disposed within the outer shaft at the first apposition state.

30. The shunting catheter system of claim 28, wherein the apposition element is disposed substantially non-symmetrically around the catheter shaft.
